(19) United States
(12) Patent Application Publication
(10) Pub. No.: US 2025/0248811 A1
(43) Pub. Date: Aug. 7, 2025

(54) IMPLANTABLE DEVICES AND SEALING MEMBERS AND ASSOCIATED METHODS

(71) Applicant: EDWARDS LIFESCIENCES CORPORATION, Irvine, CA (US)

(72) Inventors: Gillian Irene Armstrong, Long Beach, CA (US); Radhika Vaid, Irvine, CA (US)

(73) Assignee: EDWARDS LIFESCIENCES CORPORATION, Irvine, CA (US)

(21) Appl. No.: 19/190,501

(22) Filed: Apr. 25, 2025

Related U.S. Application Data
(63) Continuation of application No. PCT/US2023/080386, filed on Nov. 17, 2023.
(60) Provisional application No. 63/385,198, filed on Nov. 28, 2022.

(51) Int. Cl. A61F 2/24; A61F 2/82

(52) U.S. Cl. CPC A61F 2/2418; A61F 2/82; A61F 2210/0009; A61F 2210/0057; A61F 2210/0071; A61F 2220/0008; A61F 2230/0069; A61F 2240/001; A61F 2250/0039

(57) ABSTRACT

Sealing members for use with implantable devices and associated methods are disclosed. The sealing members include monofilament strands braided with multifilament strands into a cylindrical braided material. The monofilament strands and the multifilament strands can each comprise polyethylene terephthalate (PET). The monofilament strands can provide structural integrity and resiliency to the cylindrical braided material. The monofilament strands can provide bulk, coverage, and softness to the cylindrical braided material. A method for creating the cylindrical braided material can include a first heat setting step on a straight mandrel at a lower temperature and a second heat setting step at a higher temperature on a shaped mandrel. The sealing members made from the cylindrical braided materials can function to anchor or engage an implantable device to surrounding tissue and limit or prevent paravalvular leakage or other fluid flow around the implantable device when implanted.

### CROSS REFERENCE TO RELATED APPLICATIONS

This application is a continuation of PCT Application No. PCT/US2023/080386, filed Nov. 17, 2023, which claims the benefit of U.S. Provisional Application No. 63/385,198, filed Nov. 28, 2022, the entire contents of each of which are incorporated herein by reference.

### FIELD

The present disclosure concerns examples of implantable device and sealing members for implantable devices, as well as methods associated therewith.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

In some examples, anchoring or docking devices can be used in conjunction with expandable prosthetic valves at a native valve annulus (for example, a native mitral and/or tricuspid valve annulus), in order to more securely implant and hold the prosthetic valve at the implant site. Such anchoring/docking devices can, for example, provide a stable anchoring site, landing zone, or implantation zone at the implant site in which prosthetic valves can be expanded or otherwise implanted. M any of the disclosed docking devices comprise a coil member that can be moved into a circular or coiled or cylindrically-shaped configuration, which can (for example) allow a prosthetic heart valve comprising an annular or cylindrically-shaped valve frame or stent to be expanded or otherwise implanted into native locations with naturally circular cross-sectional profiles and/or in native locations with naturally with non-circular cross-sections.

In some examples, in addition to providing an anchoring site for the prosthetic valve, the anchoring/docking devices can be sized and shaped to cinch or draw the native valve (for example, mitral, tricuspid, etc.) anatomy radially inwards. In this manner, one of the main causes of valve regurgitation (for example, functional mitral regurgitation), specifically enlargement of the heart (for example, enlargement of the left ventricle, etc.) and/or valve annulus, and consequent stretching out of the native valve (for example, mitral, etc.) annulus, can be at least partially offset or counteracted. Some examples of the anchoring or docking devices further include features which, for example, are shaped and/or modified to better hold a position or shape of the docking device during and/or after expansion of a prosthetic valve therein. By providing such anchoring or docking devices, replacement valves can be more securely implanted and held at various valve annuluses, including, for example, at the mitral valve annulus which does not have a naturally circular cross-section.

For each of the foregoing exemplary implantable devices (and/or other types of implantable devices) the unique geometry and sometimes irregular size of a patient's native anatomy present challenges to providing an implantable prosthetic device that fits within and is provided in intimate contact or seal with the surrounding tissue when implanted.

### SUMMARY

Described herein are exemplary sealing members and exemplary implantable devices that include a sealing member (such as, for example, prosthetic heart valves, docking devices, and stents that include sealing members), as well as exemplary delivery apparatus that can be utilized with implantable devices and exemplary methods associated with the sealing members, the implantable devices, and/or the delivery apparatus.

In some examples, the sealing members disclosed herein can be comprised of a cylindrical material including multifilament strands braided with monofilament strands.

In some examples, the multifilament strands are multifilament polyethylene terephthalate (PET) strands. In some examples, the monofilament strands are monofilament PET strands.

In some examples, the monofilament strands can provide structural integrity and/or resiliency to the cylindrical braided material. In some examples, the monofilament strands of the cylindrical braided material can enable the sealing member to transition from a compressed, axially elongated configuration to an expanded, axially shortened configuration. In some examples, the monofilament strands are shape-set to the expanded, axially shortened configuration. In some examples, the expanded, axially shortened configuration is a relaxed state of the sealing member. In some examples, the monofilament strands are biased toward the expanded, axially shortened configuration.

In some examples, the multifilament strands can provide bulk to the cylindrical braided material. In some examples, the multifilament strands can provide softness to the cylindrical braided material. In some examples, the multifilament strands can provide coverage to the cylindrical braided material. In some examples, the multifilament strands of the cylindrical braided material can enable the sealing member to engage or seal a prosthetic implant to surrounding native tissue. In some examples, the multifilament strands are configured the limit or resist flow of fluid between an implantable device and the native tissue. In some examples, the multifilament strands are configured the cushion native tissue from a force exerted thereon by an implantable device.

In some examples, the sealing member can be a single layer braided cylindrical material. In some examples, the single-layer cylindrical braided material can be configured to provide structural integrity and bulk for the sealing member. In some examples, the single-layer cylindrical braided material can be configured to provide resiliency and softness for the sealing member.

In some examples, the cylindrical braided material can include an interior liner, such as a thermoplastic polyurethane (TPU) liner.

In one representative example, an implantable device comprises: an annular frame comprising a plurality of interconnected struts; and a sealing member disposed on an exterior surface of the annular frame, the sealing member comprising a plurality of monofilament strands braided with a plurality of multifilament strands to form a cylindrical braided material.

In another representative example, an implantable docking device configured for securing a prosthetic valve comprises: a coil member configured to be transitioned from a delivery configuration to a coiled configuration, wherein the coil member, when in the coiled configuration, comprises a plurality of helical turns; and a sealing member extending, relative to a central longitudinal axis of the coil member in the coiled configuration, circumferentially over at least a portion of each of one or more of the helical turns, wherein the sealing member comprises a plurality of monofilament strands braided with multifilament strands to form a cylindrical braided material.

In another representative example, a sealing member for use with an implantable device comprises a cylindrical braided material comprising a plurality of monofilament PET strands braided with multifilament PET strands.

In yet another representative example, a method of generating a sealing member comprises: loading a multifilament PET thread and a monofilament PET thread into a braider apparatus, setting one or more braiding parameters of the braider apparatus, generating a cylindrical braided material on a first mandrel comprising the multifilament PET thread braided with the monofilament PET thread, incubating the cylindrical braided material on the first mandrel at a first temperature for a first period of time, transferring the cylindrical braided material to a second mandrel, incubating the cylindrical braided material at a second temperature for a second period of time, and removing the cylindrical braided material from the second mandrel.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5A is a perspective view of an exemplary delivery apparatus and a prosthetic heart valve that can be used in the exemplary mitral valve replacement procedure illustrated in FIG. 1-4.

FIG. 5B is a side view of an exemplary delivery apparatus and a docking device that can be used in the exemplary mitral valve replacement procedure illustrated in FIG. 1-4.

FIG. 6 is a perspective view of an exemplary docking device including a sealing member, according to an example.

FIG. 7 is a perspective view of an exemplary prosthetic heart valve including a sealing member, according to an example.

FIG. 8 is a perspective view of an exemplary stent including a sealing member, according to an example.

FIG. 9A is a side view of an exemplary cylindrical braided material that can be utilized as a sealing member, according to an example.

FIG. 9B is a schematic illustration of the exemplary cylindrical braided material of FIG. 9A.

FIGS. 10A and 10B are side views of an exemplary monofilament strand and an exemplary multifilament strand, respectively, that can be utilized in generation of a cylindrical braided material.

FIGS. 11A-11E are schematic illustrations of exemplary braid patterns that can be utilized in generation of a cylindrical braided material.

FIGS. 12-15 are logical flow diagrams of exemplary methods that can be utilized for generation of a cylindrical braided material.

FIGS. 16-19 are perspective views of exemplary cylindrical braided materials at various stages during generation thereof using the exemplary methods of FIGS. 12-15.

FIGS. 20A and 20B are side and top perspective views of another exemplary cylindrical braided material including an interior liner.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

As discussed above, the unique geometry and sometimes irregular size of a patient's native anatomy (such as, for example, a native heart valve) present challenges to providing an implantable prosthetic device that fits within and is provided in intimate contact or seal with the surrounding tissue when implanted. A sealing member can be disposed on or attached to an exterior of an implantable device and be configured to engage native tissue surrounding or adjacent to the implantable device when implanted and can function to, for example, reduce regurgitation and/or promote tissue ingrowth between the native tissue and the implant. The sealing member can be configured to be in a compressed, axially elongated configuration during delivery of the implantable device to a target location (such as, for example, a native heart valve) and to transition to an expanded, axially shortened configuration during implant of (for example, radial expansion of) the implantable device at the target location.

Conventional sealing members normally include at least a two layer structure, where a first layer is comprised of a first material configured to provide structural integrity and a second layer is comprised of a second material configured to provide bulk or softness, and the two layers attached together after production of each of the first and second layers. For example, the second layer can be aligned with and inserted or disposed within the first layer, and the aligned first and second layers can be attached via suturing. The first material can be a memory-shape material (for example, nitinol) that provides the resilient structure for the seal member, while the second material can be a softer material configured to provide the bulk or density to the seal member for filling in recessed areas of the native anatomy and limiting flow of blood (or creating a seal) between the prosthetic implant and the surrounding tissue.

However, there are issues with manufacturing and utilizing the foregoing conventional sealing members. For example, as the first and second layers must be viewed under a microscope for attachment, and the steps of aligning the first and second layers and suturing the first and second layers can be difficult to perform. Further, the attachment and suturing of the first and second layers can undesirably increase production time and complexity for an implantable device including the sealing member. Y et further, the attachment and suturing of the first and second layers can be source of failure for the sealing member if the two layers are misaligned and/or if the suturing does not sufficiently attach the two layers. Further still, the attachment and suturing of the first and second layers, as well as testing of the individual layers can increase quality control and/or production costs of the implantable device.

Disclosed herein are various systems, apparatus, methods, etc., including exemplary sealing members, which can be used in conjunction with or can be a component of an implantable prosthetic device. In some examples, the exemplary sealing members disclosed herein can be utilized as an outer skirt (and/or inner skirt) for expandable prosthetic valve. In some examples, the exemplary sealing members disclosed herein can be utilized as a paravalvular leakage (PVL) guard for a docking device. In some examples, the exemplary sealing members disclosed herein can be utilized with other expandable implantable devices, such as, for example, a stent. The disclosed sealing members can function to securely seal and/or engage the prosthetic implant at the implant site, which can be, for example, a native valve annulus (for example, a native mitral and/or tricuspid valve annulus) or a vein or an artery. Further, the disclosed sealing members can be configured to transition from an axially elongated configuration (when the implantable device is a radially compressed, delivery state) to a an axially shortened and expanded configuration (when the implantable device is in a radially expanded, implanted state).

The disclosed implantable prosthetic devices, sealing members, and methods can, among other things, overcome one or more of the deficiencies of conventional implantable prosthetic devices and sealing members. For example, the sealing members disclosed herein can be comprised of a cylindrical material comprising multifilament polyethylene terephthalate (PET) strands braided with monofilament PET strands. In some examples, the sealing member can be a single layer cylindrical material comprised of the braided multifilament and monofilament PET strands. In some examples, the cylindrical material can include an interior liner, such as a liner comprising thermoplastic polyurethane (TPU), fluorinated ethylene propylene (FEP), siloxanes, acrylic, or combinations thereof, that can aid in maintaining the braided structure of the cylindrical material. As used herein, “strands” can refer to threads, yarns, strings, fibers, etc.

The monofilament PET strands can each comprise a single filament that is relatively thicker, while the multifilament PET strands can comprise a plurality of thinner filaments that are interlaced (for example, woven, wound, or braided) together to form a strand or yarn.

In some examples, the monofilament strands can provide structural integrity and/or resiliency to the cylindrical braided material. In some examples, the monofilament strands of the cylindrical material can enable the sealing member to transition from a compressed, axially elongated configuration to an expanded, axially shortened configuration. In some examples, the monofilament strands are shape-set to the expanded, axially shortened configuration. In some examples, the expanded, axially shortened configuration is a relaxed state of the sealing member. In some examples, the monofilament strands are biased toward the expanded, axially shortened configuration.

In some examples, the multifilament strands can provide bulk, softness, and/or coverage to the cylindrical braided material. In some examples, the multifilament strands of the cylindrical structure can enable the sealing member to engage or seal a prosthetic implant to surrounding native tissue. In some examples, the multifilament strands are configured the limit or resist flow of fluid between an implantable device and the native tissue. In some examples, the multifilament strands are configured the cushion native tissue from a force exerted thereon by an implantable device.

In some examples, the sealing member can be a single-layer braided cylindrical material. In some examples, the single layer cylindrical braided material can be configured to provide structural integrity and bulk for the sealing member. In some examples, the single-layer cylindrical braided material can be configured to provide resiliency and softness for the sealing member. In some examples, the cylindrical braided material can additionally include an interior liner, such as a thermoplastic polyurethane (T PU) liner.

Accordingly, the sealing members disclose herein can be more easily constructed than conventional sealing members, and have reduced failure and/or production costs relative to conventional sealing members.

### Exemplary Implantable Devices and Delivery Apparatus

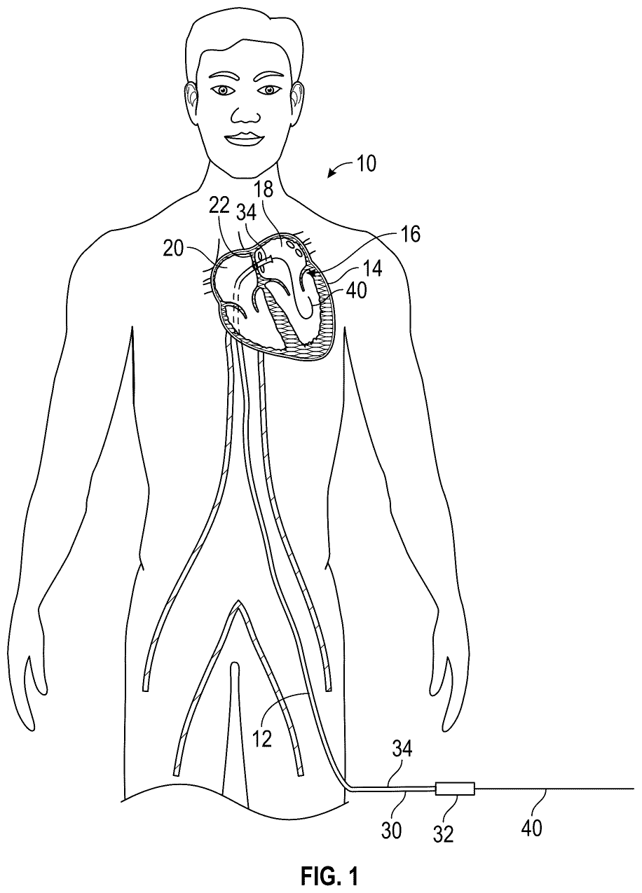

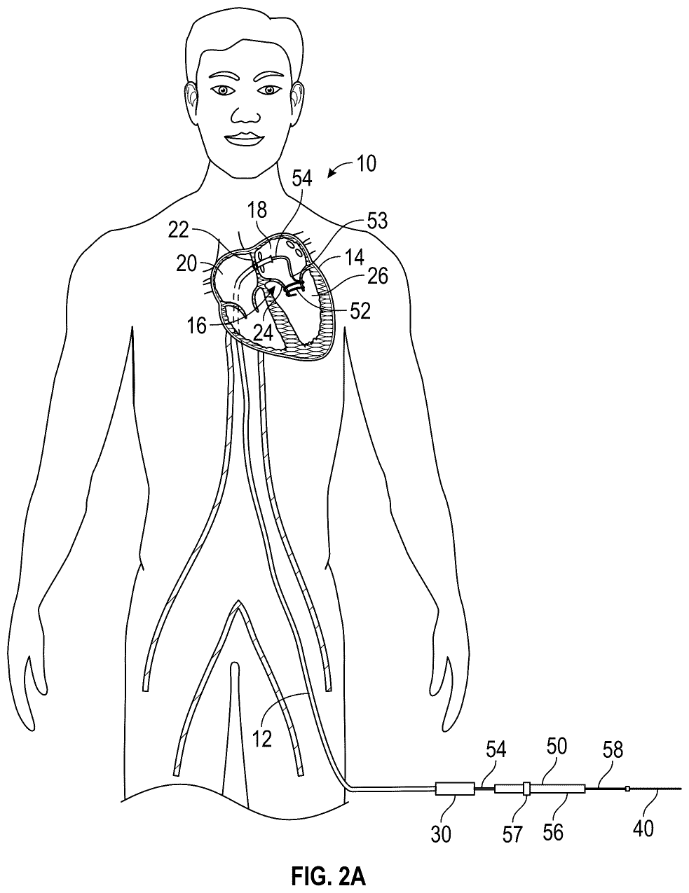

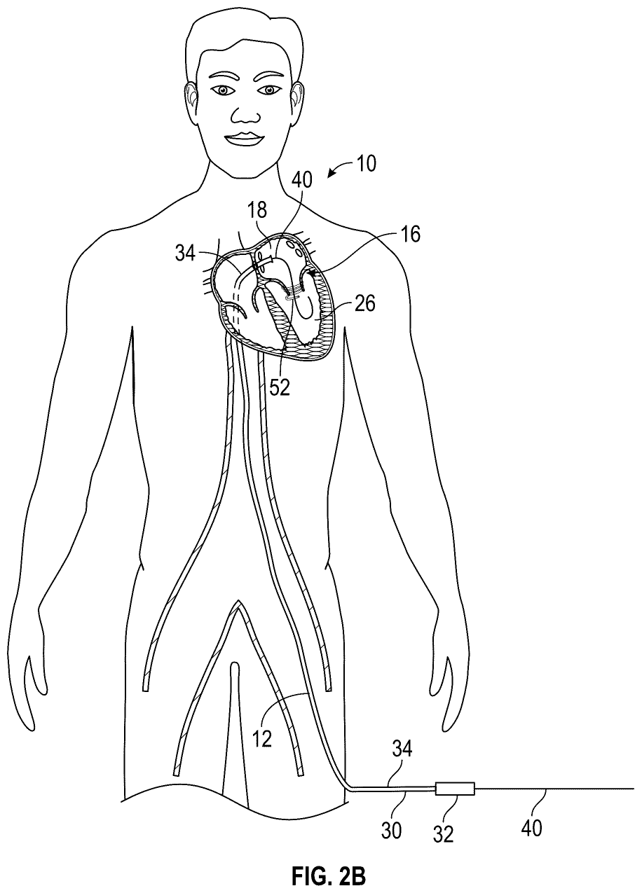

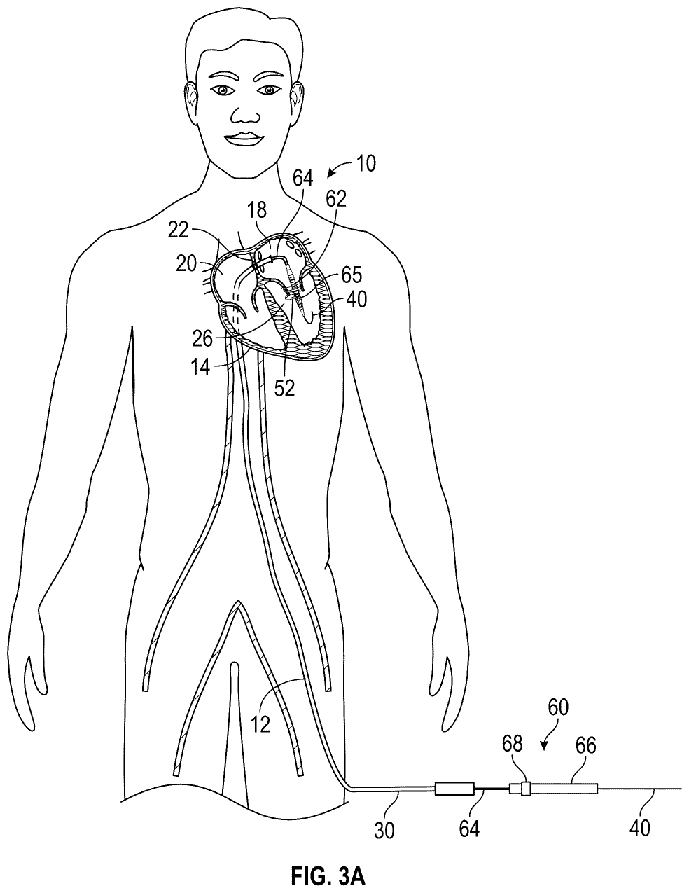

FIGS. 1-4 depict an exemplary transcatheter heart valve replacement procedure (for example, a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

Specifically, FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device”, “introducer”, or “guide sheath”) is configured to facilitate the percutaneous introduction of various implant delivery devices (for example, the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (for example, native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (for example, blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (for example, the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Patent Application Publication No. WO2020/247907, which is incorporated by reference herein in its entirety.

Referring again to FIG. 2A, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2A that is configured to be positioned within a left ventricle 26 and/or on the ventricular side of the native mitral valve 16), the user may then deploy the remaining portion of the docking device 52 (for example, an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or revolutions) of a coil that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter prosthetic heart valve” or “THV” for short, “replacement heart valve,” “prosthetic mitral valve,” and/or “prosthetic valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the coiled docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In some examples, the prosthetic heart valve 62 can be self-expanding and can be configured to radially expand on its own upon removable of a sheath or capsule covering the radially compressed prosthetic heart valve 62 on the distal end portion of the delivery shaft 64. In some examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. M ore specifically, the user can advance the delivery shaft 64 of the prosthetic valve delivery apparatus 60 by gripping and exerting a force on (for example, pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the coiled docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage (PVL) around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

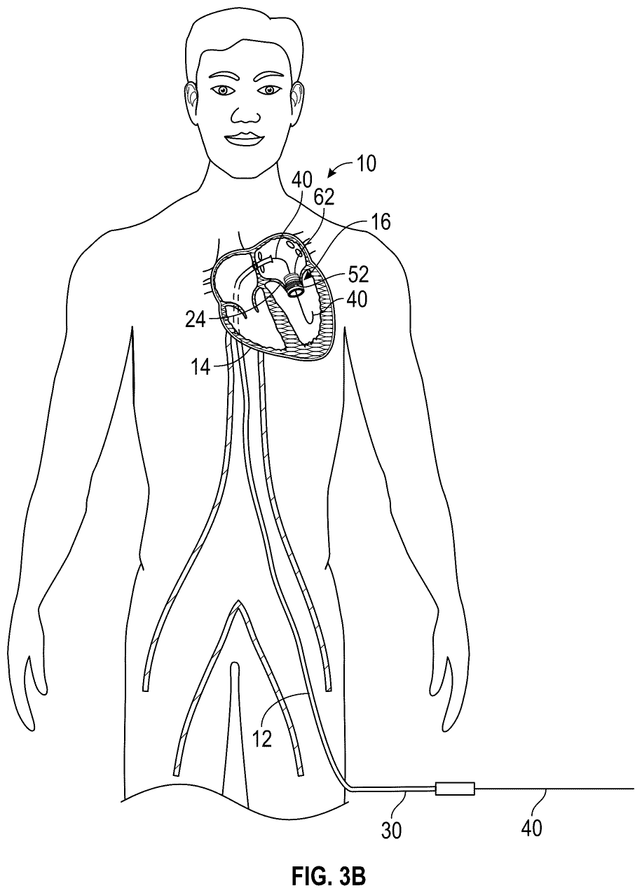

FIG. 5A illustrates an exemplary prosthetic heart valve delivery apparatus 100 (which can also be referred to here as an “implant catheter”) that can be used in lieu of the prosthetic valve delivery apparatus 60 of FIG. 3A to implant an expandable prosthetic heart valve, such as the prosthetic valves 62 or 350 described herein. In some examples, the delivery apparatus 100 is specifically adapted for use in introducing a prosthetic heart valve into a heart.

The delivery apparatus 100 in the illustrated example of FIG. 5A is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. In some examples, the delivery apparatus 100 can further comprise an inner shaft extending distally from the handle 102 coaxially through the intermediate shaft 106 and the outer shaft 104 and proximally from the handle 102 coaxially through the intermediate shaft.

The outer shaft 104 and the intermediate shaft 106 can be configured to translate (for example, move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion that extends proximally from a proximal end of the handle 102, to an adaptor 112. The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

In some examples, the intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft can extend distally beyond the distal end portion of the intermediate shaft 106 toward or to a nose cone 122 at a distal end of the delivery apparatus 100.

In some examples, a distal end of the balloon 118 can be coupled to a distal end of the delivery apparatus 100, such as to the nose cone 122 (as shown in FIG. 5A), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 (shown in FIG. 5A) can overly a distal shoulder of the delivery apparatus 100. As shown in FIG. 5A, a prosthetic heart valve 62 can be mounted around the balloon 118, at the valve mounting portion 124 of the delivery apparatus 100, in a radially compressed state. The prosthetic heart valve 62 can be configured to be radially expanded by inflation of the balloon 118 at a native valve annulus, as described above with reference to FIGS. 3A and 3B.

A balloon shoulder assembly of the delivery apparatus 100, which includes the distal shoulder, is configured to maintain the prosthetic heart valve 62 (or other medical device) at a fixed position on the balloon 118 during delivery through the patient's vasculature.

The outer shaft 104 can include a distal tip portion 128 mounted on its distal end. In some examples, the outer shaft 104 and the intermediate shaft 106 can be translated axially relative to one another to position the distal tip portion 128 adjacent to a proximal end of the valve mounting portion 124, when the prosthetic valve 62 is mounted in the radially compressed state on the valve mounting portion 124 (as shown in FIG. 5A) and during delivery of the prosthetic valve to the target implantation site. As such, the distal tip portion 128 can be configured to resist movement of the prosthetic valve 62 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Additional features of the delivery apparatus 100 that can be utilized with the prosthetic valve delivery apparatus, systems, and methods disclosed herein are described in U.S. Provisional Patent Application 63/366,897 filed on Jun. 23, 2022, which is incorporated by reference herein.

FIG. 5B illustrates a docking device delivery apparatus 200 that can be configured to implant a docking device, such as the docking devices 52 or 240 described herein or other docking devices, and that can be used in lieu of the docking device delivery apparatus 50 of FIG. 2A. In some examples, the docking device delivery apparatus 200 is specifically adapted for use in introducing a docking device into a heart. The docking device delivery apparatus can also be referred to as a “dock delivery catheter” or “dock delivery system.”

As shown, the delivery apparatus 200 can include a handle assembly 202 and a delivery sheath 204 (also referred to as the “delivery shaft” or “outer shaft” or “outer sheath”) extending distally from the handle assembly 202. The handle assembly 202 can include a handle 206 including one or more knobs, buttons, wheels, and/or other means for controlling and/or actuating one or more components of the delivery apparatus 200. For example, in some examples, as shown in FIG. 5B, the handle 206 can include knobs 208 and 210 which can be configured to steer or control flexing of components of the delivery apparatus 200.

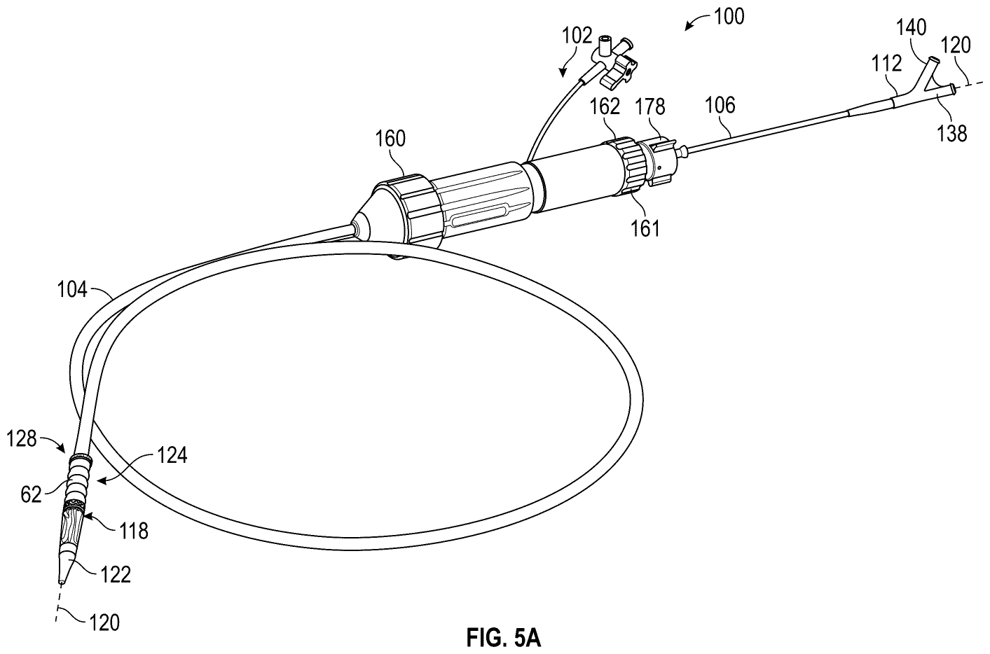

In some examples, the delivery apparatus 200 can also include a pusher shaft 212 and/or a sleeve shaft (not shown), both of which can extend through an inner lumen of the delivery sheath 204 and have respective proximal end portions extending into the handle assembly 202. A distal end portion (also referred to as “distal section”) of the sleeve shaft can include a lubricous dock sleeve configured to cover or surround the docking device 52, 300. For example, as shown in FIG. 7B and further discussed below, a docking device 300 can be retained or disposed inside of a dock sleeve 220. In some examples, the docking device 300 within the dock sleeve 220 can be further retained by or disposed within a distal end portion 205 of the delivery sheath 204, when navigating through a patient's vasculature.

Additionally, the distal end portion 205 of the delivery sheath 204 can be configured to be steerable. In one example, by rotating a knob (for example, 208 or 210) on the handle 206, a curvature of the distal end portion 205 can be adjusted so that the distal end portion 205 of the delivery sheath 204 can be oriented in a desired angle. For example, to implant the docking device 52 at the native mitral valve location, the distal end portion 205 of the delivery sheath 204 can be steered in the left atrium so that the dock sleeve 220 and the docking device 52 retained therein can extend through the native mitral valve annulus at a location adjacent the posteromedial commissure.

In some examples, the pusher shaft 212 and the sleeve shaft (not shown) can be coaxial with one another, at least within the delivery sheath 204. In addition, the delivery sheath 204 can be configured to be axially movable relative to the sleeve shaft and the pusher shaft 212. As described further below, a distal end of the pusher shaft 212 can be inserted into a lumen of the sleeve shaft and press against the proximal end of the docking device 52 retained inside the dock sleeve 220.

After reaching a target implantation site, the docking device 52 can be deployed from the delivery sheath 204 by manipulating the pusher shaft 212 and sleeve shaft using a hub assembly 218, as described further below. For example, by pushing the pusher shaft in the distal direction while holding the delivery sheath 204 in place or retracting the delivery sheath 204 in the proximal direction while holding the pusher shaft in place, or pushing the pusher shaft 212 in the distal direction while simultaneously retracting the delivery sheath 204 in the proximal direction, the docking device 52 can be pushed out of a distal end 204d of the delivery sheath 204, thus changing from the delivery configuration to the deployed configuration. In some examples, the pusher shaft 212 and the sleeve shaft can be actuated independently of each other.

In some examples, when deploying the docking device 52 from the delivery sheath 204, the pusher shaft 212 and the sleeve shaft can be configured to move together with the docking device 52 in the axial direction. For example, actuation of the pusher shaft 212, to push against the docking device 52 and move it out of the delivery sheath 204 can also cause the sleeve shaft to move along with the pusher shaft 212 and the docking device 52. As such, the docking device 52 can remain covered by the dock sleeve 220 of the sleeve shaft during the procedure of pushing the docking device 52 into position at the target implantation site via the pusher shaft 212. Thus, in some examples, when the docking device 52 is initially deployed at the target implantation site, the lubricous dock sleeve 220 can facilitate the covered docking device 52 encircling the native anatomy.

During delivery, the docking device 52 can be coupled to the delivery apparatus 200 via a release suture 214 (or other retrieval line comprising a string, yarn, or other material that can be configured to be tied around the docking device 52 and cut for removal) that extends through the pusher shaft 212. In one specific example, the release suture 214 can extend through the delivery apparatus 200, for example, through an inner lumen of the pusher shaft 212, to a suture lock assembly 216 of the delivery apparatus 200.

The handle assembly 202 can further include a hub assembly 218 to which the suture lock assembly 216 and a sleeve handle 224 are attached. The hub assembly 218 can be configured to independently control the pusher shaft 212 and the sleeve shaft while the sleeve handle 224 can control an axial position of the sleeve shaft relative to the pusher shaft 212. In this way, operation of the various components of the handle assembly 202 can actuate and control operation of the components arranged within the delivery sheath 204. In some examples, the hub assembly 218 can be coupled to the handle 206 via a connector 226.

The handle assembly 202 can further include one or more flushing ports (for example, three flushing ports 232, 236, 238 are shown in FIG. 5B) to supply flush fluid to one or more lumens arranged within the delivery apparatus 200 (for example, annular lumens arranged between coaxial components of the delivery apparatus 200).

Further details on delivery apparatus/catheters/systems (including some examples of the handle assembly) that are configured to deliver a docking device to a target implantation site can be found in U.S. Patent Application Publication Nos. 2018/0318079 and 2018/0263764, and PCT Patent Application No. US2021/056150, which are each incorporated by reference herein.

In some examples, one or more of the foregoing delivery techniques and methods are carried out in a simulation procedure, which are not conducted on a living human body. For example, the methods and techniques can be performed on a model anatomical system, in a cadaver, or in an animal.

### Exemplary Implantable Devices and Sealing Members

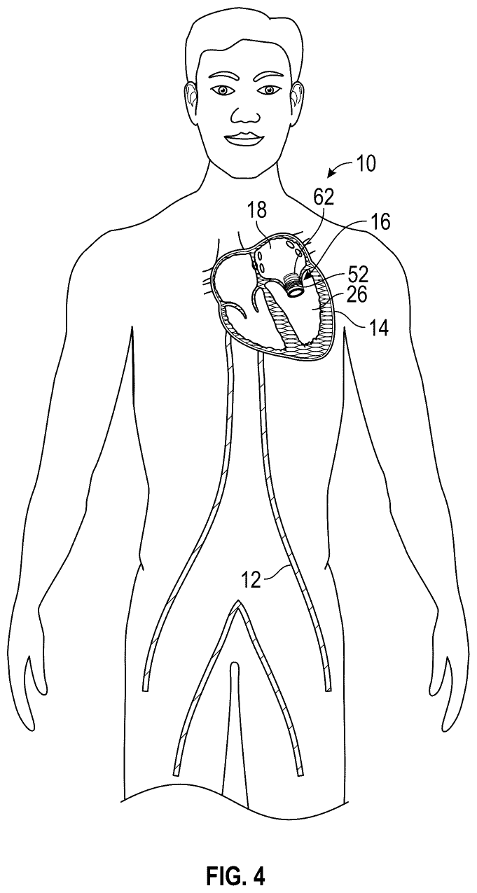

Turning now to FIGS. 6-8 exemplary implantable devices including a sealing member are illustrated and described. In one example, FIG. 6 shows a docking device 240 including a sealing member 244. The docking device 240 can, for example, be used as the docking device 52 in a prosthetic valve implantation procedure, as described above with reference to FIGS. 1-4 and 5B. The docking device in its deployed configuration (as illustrated in FIG. 6) can be configured to receive and secure a prosthetic valve within the docking device, and thereby securing the prosthetic valve at the native valve annulus when implanted.

The docking device 240 can comprise a coil member 242 having the sealing member 244 disposed therearound and covering at least a portion of the coil member 242. In some examples, the coil member 242 can include a shape memory material (for example, nickel titanium alloy or “Nitinol”) such that the docking device 240 (and the coil member 242) can move from a substantially straight configuration (or delivery configuration) when disposed within the delivery sheath 204 of the delivery apparatus 200 to a helical, deployed configuration after being removed from the delivery sheath 204.

The coil member 242 has a proximal end 242p and a distal end 242d (which also respectively define the proximal and distal ends of the docking device 240). When being disposed within the delivery sheath 204 (for example, during delivery of the docking device 240 into the vasculature of a patient), a body of the coil member 242 between the proximal end 242p and distal end 242d can form a generally straight delivery configuration (i.e., without any coiled or looped portions, but can be flexed or bent) so as to maintain a small radial profile when moving through a patient's vasculature. After being removed from the delivery sheath 204 and deployed at an implant position, the coil member 242 can move from the delivery configuration to the helical deployed configuration and wrap around native tissue adjacent the implant position. For example, when implanting the docking device at the location of a native valve, the coil member 242 can be configured to surround native leaflets of the native valve (and the chordae tendineae that connects native leaflets to adjacent papillary muscles, if present).

The docking device 240 can be releasably coupled to the delivery apparatus 200. For example, in certain examples, the docking device 240 can be coupled to a delivery apparatus (as described above) via a release suture that can be configured to be tied to the docking device 240 and cut for removal.

As shown in FIG. 6, the coil member 242 in the deployed configuration can include a leading turn 246 (or “leading coil”), a central region 248, and a stabilization turn 250 (or “stabilization coil”) around a central longitudinal axis. The central region 248 can possess one or more helical turns having substantially equal inner diameters. The leading turn 246 can extend from a distal end of the central region 248 and has a diameter greater than the diameter of the central region 248, in the illustrated example. The stabilization turn 250 can extend from a proximal end of the central region 248 and has a diameter greater than the diameter of the central region 248, in the illustrated example. In some examples, the stabilization turn 250 can be omitted from the coil member 242, for example, when another retention member is used to stabilize the positioning of the docking device 240 relative to the native anatomy during an implant procedure. Alternatively, the stabilization turn 250 can have a diameter that is equal, approximately equal, or less than the diameter of the central region 248 (as opposed to larger), and/or the stabilization turn can comprise less of a full turn than depicted in FIG. 6.

The sealing member 244 can be configured to transition between an axially elongated and/or radially compressed configuration and an axially shortened, radially expanded configuration. For example, the sealing member 244 can be in the axially elongated configuration while the coil member 242 is in the substantially straight configuration (or delivery configuration) when disposed within the delivery sheath 204 of the delivery apparatus 200. While in the axially elongated configuration, the sealing member can have a decreased diameter and/or a lower profile, thereby giving the docking device 240 an overall decreased profile during delivery thereof to the implantation site, such as via the method described with respect to FIG. 1.

After or while the coil member 242 is transitioned to the helical, deployed configuration (FIG. 6) and is positioned at the implantation site (FIGS. 2A-4), the sealing member 244 can be transitioned to the axially shortened, radially expanded configuration. When in the axially shortened configuration, the sealing member 244 is radially expanded relative to a central axis or an outer surface of the coil member 242 and can have an increased diameter or larger profile relative to the axially elongated configuration.

In some examples, the sealing member 244 can be a PVL guard member configured to seal or engage the docking device 240 with the native tissue when implanted. In some examples, the PVL guard member is configured to prevent, limit, or resist paravalvular leakage and/or reduce regurgitation between the native tissue and the docking device 240. In some examples, the PVL guard member is configured to promote tissue ingrowth between the native tissue and the docking device 240.

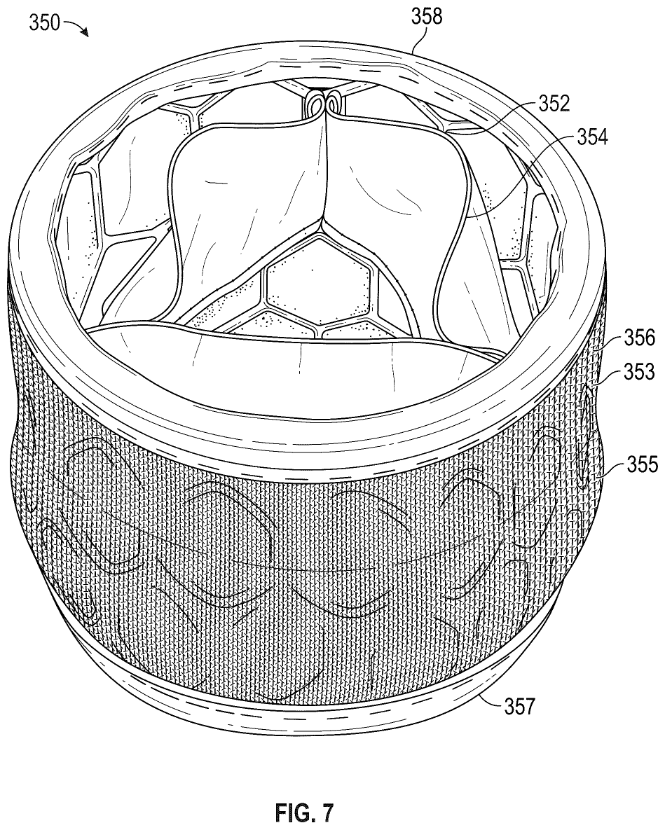

In some examples, the sealing member 244 is a cylindrical material comprising multifilament PET strands braided (see, for example, FIG. 10B) with monofilament PET strands (see, for example, FIG. 10A). In some examples, the monofilament PET strands can provide structural stability and/or resiliency to the form or shape of the sealing member 244. As can be seen in FIG. 6, in some examples, in the axially shortened (radially expanded) configuration, the sealing member 244 can have a generally tubular shape having a greatest diameter within a central region or portion 244c. Further, the sealing member can be tapered towards the proximal end 244p and the distal end 244d thereof.

In some examples, the monofilament strands in the sealing member 244 can be shape-set and/or biased toward the axially shortened (radially expanded) configuration of the sealing member. Thus, in some examples, the axially shortened (radially expanded) configuration is a relaxed configuration or state of the sealing member. This can enable the sealing member 244 to be temporarily retained in the axially elongated (radially compressed) configuration during delivery of the docking device 240, and when released or exposed from a sheath (for example, from the delivery sheath 204 or a separate delivery sheath) the sealing member 244 can transition to the axially shortened, radially expanded configuration. In some examples, the biasing force is sufficient to transition the sealing member 244 to the axially shortened (radially expanded) configuration. In some examples, an additional sheath or delivery apparatus subcomponent (for example, a pusher shaft or a puller shaft) can be configured to move the sealing member 244 into the axially shortened, radially expanded configuration by applying a force to one end thereof and moving the end relative to and/or over the surface of the coil member 242, and the biasing force can retain the sealing member 244 in the axially shortened, radially expanded configuration.

In some examples, the multifilament PET strands provide bulk, coverage, and/or softness to the sealing member 244. In some examples, the multifilament strands are each comprised of a plurality of relatively thinner PET filaments that are in a loose arrangement relative to each other. For example, the multifilament strands can have some areas of contact between the individual filaments and some areas of open space disposed between the individual filaments. In some examples, the multifilament strands can be configured to fill in recessed areas of the native anatomy. In some examples, the multifilament strands can be configured to fill in open spaces between the monofilament strands within the braided structure. In some examples, the multifilament strands can be configured to limit flow of fluid (for example, blood) between the docking device and the surrounding tissue. In some examples, the multifilament strands can be configured to cushion the native tissue from forces exerted thereon by the docking device.

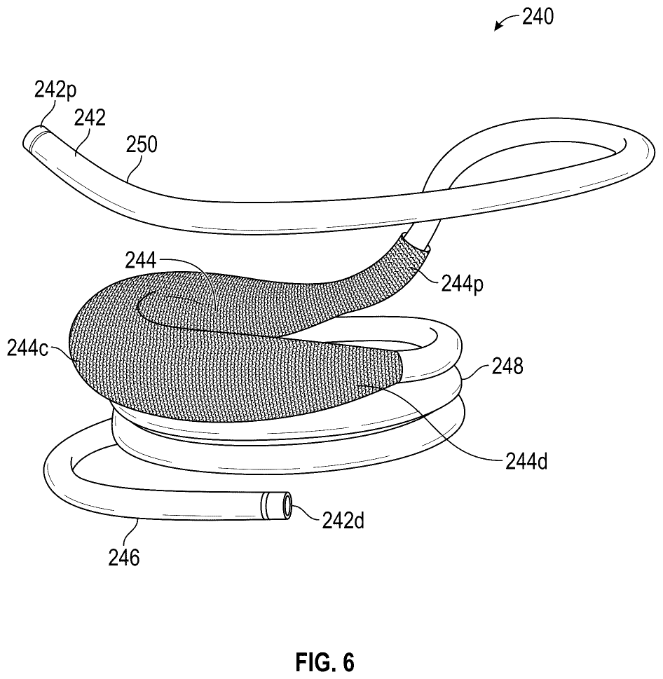

Further details of the cylindrical braided material and the monofilament and multifilament strands are discussed below with reference to FIGS. 9A-20B. Further details of the docking device and its variants are described in International Application No. PCT/US2021/056150, which is incorporated by reference herein in its entirety.

In another example, FIG. 7 illustrates a prosthetic heart valve 350 including a sealing member 356. The prosthetic valve 350 can be used as the prosthetic heart valve 62 in a prosthetic valve implantation procedure, as described above with reference to FIGS. 1-4. Any of the prosthetic valves disclosed herein can be adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

As discussed above, in some examples, the disclosed prosthetic valves can be implanted within a docking or anchoring device (for example, the docking device 240, etc.) that is implanted within a native heart valve or a vessel. In some examples, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIG. 7 shows the prosthetic valve 350 in a radially expanded state or configuration. As illustrated in FIG. 7, the prosthetic valve 350 can include a frame 352 and a plurality of leaflets 354 can be situated at least partially within the frame 352. As noted above, the prosthetic valve 350 can also include the sealing member 356 situated on and/or attached to an exterior surface the frame 352. The prosthetic valve 350 can include an inflow end 357 and an outflow end 358. The terms “inflow” and “outflow” are related to the normal direction of blood flow (for example, antegrade blood flow) through the prosthetic valve 350. For example, the leaflets 354 can allow blood flow through the valve 350 in a direction from the inflow end 357 to the outflow end 358 and prevent the reverse flow (for example, prevent flow in a direction from the outflow end 358 to the inflow end 357).

The frame 352 can be made of any of various suitable plastically-expandable materials (for example, stainless steel, etc.) or self-expanding materials (for example, Nitinol) as known in the art. When constructed of a plastically-expandable material, the frame 352 (and thus the valve 350) can be crimped to a radially compressed state on a delivery catheter (such as, delivery apparatus 100 show in FIG. 5A) and then expanded inside a patient by an inflatable balloon or equivalent expansion mechanism. When constructed of a self-expandable material, the frame 352 (and thus the valve 350) can be crimped to a radially compressed state or configuration and retained in the compressed state by insertion into a sheath or equivalent mechanism of a delivery catheter. Once inside the body, the valve 350 can be advanced from the delivery sheath, which allows the valve to expand to its functional size when in the radially expanded configuration.

Suitable plastically-expandable materials that can be used to form the frames disclosed herein (for example, the frame 352) include, metal alloys, polymers, or combinations thereof. Example metal alloys can comprise one or more of the following: nickel, cobalt, chromium, molybdenum, titanium, or other biocompatible metal. In some examples, the frame 352 can comprise stainless steel. In some examples, the frame 352 can comprise cobalt-chromium. In some examples, the frame 352 can comprise nickel-cobalt-chromium. In some examples, the frame 352 comprises a nickel-cobalt-chromium-molybdenum alloy, such as MP35N™ (tradename of SPS Technologies), which is equivalent to UNS R30035 (covered by A STM F562-02). MP35N™/UNS R30035 comprises 35% nickel, 35% cobalt, 20% chromium, and 10% molybdenum, by weight.

When the frame 352 (and the valve 350) are crimped to the radially compressed state, the sealing member 356 can be transitioned into an axially elongated, compressed state and retained in the axially elongated state by the insertion of the valve 350 into the sheath of the delivery catheter. When the frame 352 (and the valve 350) are transitioned to the radially expanded state (FIG. 7) and positioned at the implantation site (FIGS. 3B-4), the sealing member 356 can be transitioned to an axially shortened and/or radially expanded configuration. While in the axially shortened configuration, the sealing member 356 can radially expanded relative to a central axis of the frame 352 and have an increased diameter or larger profile relative to the axially shortened and/or radially expanded configuration.

In some examples, the sealing member 356 can be an outer covering or outer skirt, configured to seal or engage the prosthetic valve 350 with surrounding native tissue when implanted. In some examples, the outer skirt is configured to prevent, limit, or resist paravalvular leakage and/or reduce regurgitation between the native tissue and the prosthetic valve 350. In some examples, the outer skirt is configured to promote tissue ingrowth between the native tissue and the prosthetic valve 350.

In some examples, the sealing member 356 is a cylindrical material comprising multifilament PET strands (see, for example, FIG. 10B) braided with monofilament PET strands (see, for example, FIG. 10A). In some examples, the monofilament PET strands can provide structural stability and/or resiliency to the form or shape of the sealing member 356. As can be seen in FIG. 7, in some examples, in the axially shortened, radially expanded configuration, the sealing member 356 can have a generally cylindrical shape that includes a non-bulging portion 353 and a bulging portion 355. In such examples, the sealing member can have a greatest diameter at the bulging portion 355 and a smaller diameter at the non-bulging region 353. In some examples (as illustrated in FIG. 7), the non-bulging portion 353 can be oriented toward the outflow end 358 and the bulging portion 355 can be oriented toward the inflow end 357. Although not specifically illustrated, in some examples, the non-bulging portion 353 can be oriented toward the inflow end 357 and the bulging portion 355 can be oriented toward the outflow end 358.

In some examples, the monofilament strands in the sealing member 356 can be shape-set in and/or biased toward the axially shortened, radially expanded configuration of the sealing member. Thus, in some examples, the axially shortened, radially expanded configuration is a relaxed configuration or state of the sealing member. This can enable the sealing member 356 to be temporarily retained in the axially elongated, radially compressed configuration during delivery of the prosthetic valve 350, and when released or exposed from a sheath of a valve delivery apparatus (for example, the delivery apparatus 100) the sealing member 356 can transition to the axially shortened, radially expanded configuration.

In some examples, the multifilament PET strands provide bulk, coverage, and/or softness to the sealing member 356. In some examples, the multifilament strands are comprised of a plurality of relatively thinner PET filaments that are in a loose arrangement relative to each other. For example, the multifilament strands can have some areas of contact between the individual filaments and some areas of open space disposed between the individual filaments. In some examples, the multifilament strands can be configured to fill in recessed areas of the native anatomy. In some examples, the multifilament strands can be configured to fill in open spaces between the monofilament strands within the braided structure. In some examples, the multifilament strands can be configured to limit flow of fluid (for example, blood) between the prosthetic valve and the surrounding tissue. In some examples, the multifilament strands can be configured to cushion the native tissue from forces exerted thereon by the prosthetic valve.

Further details of the cylindrical braided material and the monofilament and multifilament strands are discussed below with reference to FIGS. 9A-20B. Further details of the prosthetic heart valves, as well as variants and components thereof are described in U.S. Pat. Nos. 9,393,110; 9,339,384; and 11,185,406, which are each incorporated by reference herein. Additional examples of valve covers and outer skirts are described in PCT Patent Application Publication No. WO/2020/247907 and U.S. Patent Publication No. US2019/0192296, which are each incorporated by reference herein.

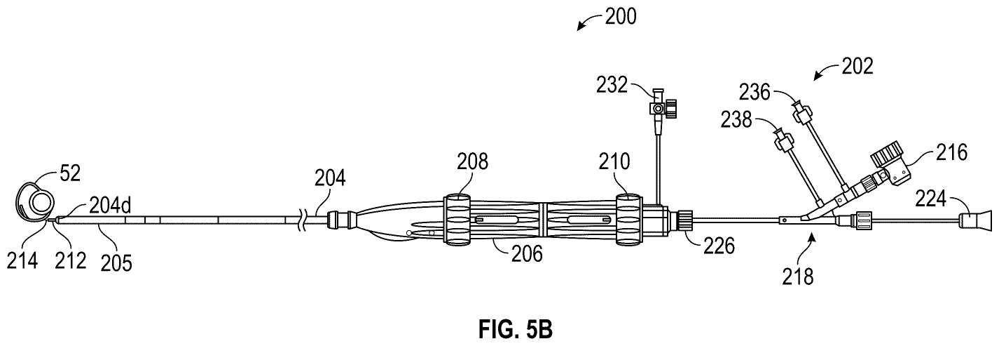

In another example, FIG. 8 illustrates a stent 450 including a sealing member 456. In some examples, the stent 450 can have a similar structure to the prosthetic heart valve 350, however, the stent 450 lacks a valvular structure or leaflets. The stent 450 can be configured for insertion into a lumen of an anatomic vessel or duct to keep the passageway open implantation. For example, stents having the configuration of the stent 450 can be implanted in one or more veins, arteries, and/or vessels of a patient. In some examples, the stent 450 can be implanted in a similar manner as the implantation method discussed above with reference to FIGS. 1-4 and/or using similar delivery apparatus as the delivery apparatus discussed above with reference to FIGS. 5A-5B. In some examples, stents having one or more features of the stent 450 (such as, the sealing member 456) can be implanted within and used for opening other anatomical lumen, such as in the kidney or bladder.

FIG. 8 shows the stent 450 in a radially expanded state or configuration. As illustrated in FIG. 8, the stent 450 can include a frame 452 and the sealing member 456 situated on an exterior surface the frame 452. The stent 450 can include a first end 457 and a second end 458, which can be defined as “inflow” or “outflow” by the normal direction of blood flow through the stent 450 when implanted in a specified orientation. Thus, in some examples, the end 457 is the outflow end and the end 458 is the inflow end, and, in other examples, the end 458 is the outflow end and the end 457 is the inflow end, depending on the orientation of implantation within the vein or artery.

Similar to the frame 352, the frame 452 can be made of any of various suitable plastically-expandable materials (for example, stainless steel, etc.) or self-expanding materials (for example, Nitinol) as known in the art. When constructed of a plastically-expandable material, the frame 452 (and thus the stent 450) can be crimped to a radially compressed state on a delivery catheter and then expanded inside a patient by an inflatable balloon or equivalent expansion mechanism. When constructed of a self-expandable material, the frame 452 (and thus the stent 450) can be crimped to a radially compressed state or configuration and retained in the compressed state by insertion into a sheath or equivalent mechanism of a delivery catheter. Once inside the body, the stent can be advanced from the delivery sheath, which allows the stent to expand to its functional size when in the radially expanded configuration.

Suitable plastically-expandable materials that can be used to form the frames disclosed herein (for example, the frame 452) include, metal alloys, polymers, or combinations thereof. Example metal alloys can comprise one or more of the following: nickel, cobalt, chromium, molybdenum, titanium, or other biocompatible metal. In some examples, the frame 452 can comprise stainless steel. In some examples, the frame 452 can comprise cobalt-chromium. In some examples, the frame 452 can comprise nickel-cobalt-chromium. In some examples, the frame 452 comprises a nickel-cobalt-chromium-molybdenum alloy, such as MP35N™ (tradename of SPS Technologies), which is equivalent to UNS R30035 (covered by A STM F562-02). MP35NTM/UNS R30035 comprises 35% nickel, 35% cobalt, 20% chromium, and 10% molybdenum, by weight.

When the frame 452 (and the stent 450) are crimped to the radially compressed state, the sealing member 456 can be transitioned into an axially elongated, compressed state and retained in the axially elongated state by the insertion of the stent 450 into the sheath of the delivery catheter. When the frame 452 (and the stent 450) are transitioned to the radially expanded state (FIG. 8) and positioned at the implantation site, the sealing member 456 can be transitioned to an axially shortened and/or radially expanded configuration. While in the axially shortened configuration, the sealing member 456 can radially expanded relative to a central axis of the frame 452 and have an increased diameter or larger profile relative to the axially shortened and/or radially expanded configuration.

In some examples, the sealing member 456 can be an outer covering or outer skirt, configured to seal or engage the stent 450 with surrounding native tissue when implanted. In some examples, the outer skirt is configured to prevent, limit, or resist flow of fluid between the native tissue and the stent 450. In some examples, the outer skirt is configured to promote tissue ingrowth between the native tissue and the prosthetic valve 450.

In some examples, the sealing member 456 is a cylindrical material comprising multifilament PET strands braided (see, for example, FIG. 10B) with monofilament PET strands (see, for example, FIG. 10A). In some examples, the monofilament PET strands can provide structural stability and/or resiliency to the form or shape of the sealing member 456. As can be seen in FIG. 8, in some examples, in the axially shortened (radially expanded) configuration, the sealing member 456 can have a generally cylindrical shape that includes a non-bulging portion 453 and a bulging portion 455. In such examples, the sealing member can have a greatest diameter at the bulging portion 455 and a smaller diameter at the non-bulging region 453.

In some examples, the monofilament strands in the sealing member 456 can be shape-set in and/or biased toward the axially shortened, radially expanded configuration of the sealing member. Thus, in some examples, the axially shortened, radially expanded configuration is a relaxed configuration or state of the sealing member. This can enable the sealing member 456 to be temporarily held in the axially elongated, radially compressed configuration during delivery of the stent 450, and when released or exposed from a sheath of a stent delivery apparatus the sealing member 456 can transition to the axially shortened, radially expanded configuration.

In some examples, the multifilament PET strands provide bulk, coverage, and/or softness to the sealing member 456. In some examples, the multifilament strands are comprised of a plurality of relatively thinner PET filaments that are in a loose arrangement relative to each other. For example, the multifilament strands can have some areas of contact between the individual filaments and some areas of open space disposed between the individual filaments. In some examples, the multifilament strands can be configured to fill in recessed areas of the native anatomy. In some examples, the multifilament strands can be configured to fill in open spaces between the monofilament strands within the braided structure. In some examples, the multifilament strands can be configured to limit flow of fluid (for example, blood) between the stent and the surrounding tissue. In some examples, the multifilament strands can be configured to cushion the native tissue from forces exerted thereon by the implanted stent.

Further details of the cylindrical braided material and the monofilament and multifilament strands are discussed below with reference to FIGS. 9A-20B. Additional examples of valve covers and outer skirts are described in PCT Patent Application Publication No. WO/2020/247907 and U.S. Patent Publication No. US2019/0192296, previously incoporated herein.

Turning now to FIGS. 9A-20B, exemplary cylindrical braided materials, monofilament PET strands, multifilament PET strands, and associated methods are described in detail.

FIG. 9A shows an exemplary cylindrical braided material or structure 900, and FIG. 9B is a schematic illustration of the cylindrical braided material 900. The cylindrical braided material 900 includes a plurality of monofilament strands 902 braided with a plurality of multifilament PET strands 904.

Similar to the sealing members 356, 456, the cylindrical braided material 900 can have a generally cylindrical shape including a non-bulging portion 953 and a bulging portion 955. As shown in FIG. 9B, the cylindrical braided material 900 can have a diameter a at a widest section of the bulging portion 955, and can have a diameter b at the non-bulging portion 953. In some examples, the diameter a is in a range of 32 mm to 34 mm. In some examples, the diameter bis in a range of 20 mm to 40 mm. In one specific example, the diameter bis 28.6 mm. Also shown in FIG. 9B, the bulging portion 955 can have a height c, the non-bulging portion 953 can have a height d, and the cylindrical braided material 900 can have an overall height e. In some examples, the height cis in a range of 10 mm to 12 mm. In some specific example the height c, is 11.3 mm. In some examples, the heights d and e are related to dimensions of a mandrel on which the cylindrical braided material 900 is formed. For example, the non-bulging portion 953 can be an excess portion of the cylindrical braided material 900 that will be removed or cut off when used to form a sealing member.

As can best be seen the schematic illustration of FIG. 9B, in some examples, the bulging portion 955 can include linear sections 958, 959 at opposing ends of the bulging portion 955, each having a height f (f₁ and f₂). In some examples, the height f is in a range of 1.0 mm to 2.0 mm. In some examples, the two linear sections 958 and 959 can have the same height (f₁=f₂). In some examples, the two linear sections 958 and 959 can have differing heights. For example, the linear section 959 can have a greater height than the linear section 958 (f₁<f₂). In an alternative example, the linear section 958 can have a greater height than the linear section 959 (f₁>f₂).

It will be appreciated that the shape and dimensions of the cylindrical braided material 900 are exemplary, and a cylindrical braided material can have various shapes and dimensions and are configured to enable coupling to or use with a specific implantable device. For example, a cylindrical braided material (such as, for example, a cylindrical braided material for the sealing member 244 shown in FIG. 6) can have a shape including a central portion (for example, central portion 244c) having a greatest diameter, and a width of the cylindrical braided material can decrease as the overall shape tapers towards the ends thereof (for example, ends 244p, 244d). In another example, a cylindrical braided material for an alternative sealing member on a prosthetic valve can include two or more bulging portions, for example, where it is desired for an outer skirt to include a bulging portion to extend over each of the inflow side and the outflow side of a native annulus when the prosthetic valve is implanted therein. Various other configuration and shapes for a cylindrical braided material can be achieved via the examples and methods disclosed herein.

FIGS. 10A and 10B, show exemplary schematic illustrations of a monofilament PET strand 902 (FIG. 10A) and a multifilament PET strand 904 (FIG. 10B). As can be seen therein, the monofilament PET strand 902 can comprise a single thicker filament, while the multifilament PET strand 904 can comprise a plurality of thinner filaments 906 that are interlaced (for example, twisted) together to form the strand or yarn.

In some examples, the monofilament PET strand 902 (as illustrated in FIG. 10A) has a larger diameter than the overall diameter of the multifilament PET strand of FIG. 10B. For example, for sealing members where increased structural stability is desired, such as in the sealing member 244, a cylindrical braided material can include monofilament PET strands having a greater diameter than the overall diameter of the multifilament PET strands. In some examples, the multifilament PET strands can have a greater overall diameter than the monofilament strand. For example, for sealing members where increased softness and/or coverage is desired, such as in the sealing members 356, 456, a cylindrical braided material can include multifilament PET strands having a greater overall diameter than a diameter of the monofilament PET strands. It will be appreciated that the diameter of each of the monofilament and multifilament PET strands can be selected based on structural requirements or desired structural characteristics of a sealing member.

In some examples, a thickness or diameter or gauge of the PET material in the monofilament strand 902 can be in the range of 0.002 in to 0.015 in. In one specific example, the monofilament PET strand is 0.004 in PET. In some examples, the monofilament strand can comprise a different material. For example, the monofilament strand can comprise PEEK, TPU, polyesters (such as, PLA, P4HB, PGA, PCL, etc), polyamides, UHM WPE (Polyethylenes), or polypropylene. In some examples, the monofilament strand 902 can be round. In some examples, the monofilament strand 902 can be flat.

In some examples, a thickness or diameter of gauge of the individual PET filaments in the multifilament strand 904 can be the range of 0.002 mm to 0.005 mm, and an overall thickness or diameter the multifilament PET strand 904 can be in the range of 0.0015 in to 0.004 in. In one specific example, the multifilament PET strand is 140 Denier/68f/Z Textured PET. In some examples, the multifilament strand can comprise a different material. For example, the multifilament strand can comprise High Tenacity PET, polyesters (such as, PLA, P4HB, PGA, etc), polyamides, UHMWPE (polyethylenes), polypropylene.

In some examples, the multifilament strand is texturized. For example, the individual filaments of a multifilament strand can be crimped or looped to provide bulk and texture to the final thread or multifilament structure. Texturized filaments enable the multifilament strands to have a greater volume than multifilament threads which include flat individual filaments (that is, filaments that have not undergone the texturizing process) of the same size and filament count.

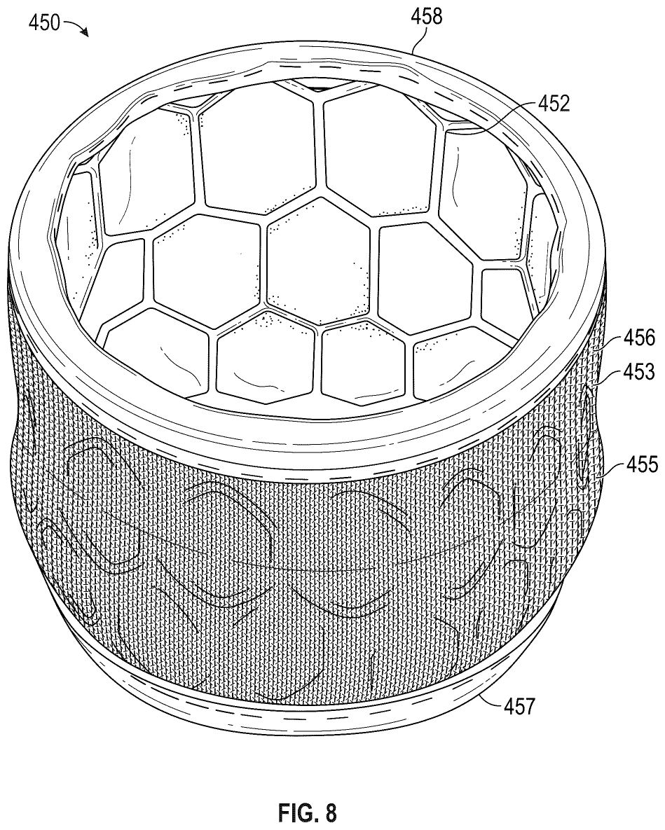

The monofilament strands 902 can be braided with the multifilament strands 904 in a specified or selected braiding pattern. FIGS. 11A-11E show exemplary braiding patterns that can be utilized for braiding of the monofilament and multifilament strands 902, 904 into a cylindrical braided material. FIG. 11A illustrates a regular, one up-two down braid pattern. FIG. 11B illustrates a Hercules, three up-three down braid pattern. FIG. 11C illustrates a diamond or full load, two up-two down braid pattern. FIG. 11D illustrates a half diamond, one up-one down braid pattern. FIG. 11E illustrates a half diamond braid pattern including a triaxial thread. It will be appreciated that the braid patterns disclosed herein are merely exemplary and other braid patterns and variations on the illustrated braid patterns can be utilized to generate a monofilament and multifilament cylindrical braided material for a sealing member.

### Exemplary Monofilament and Multifilament Cylindrical Braided Materials and Methods

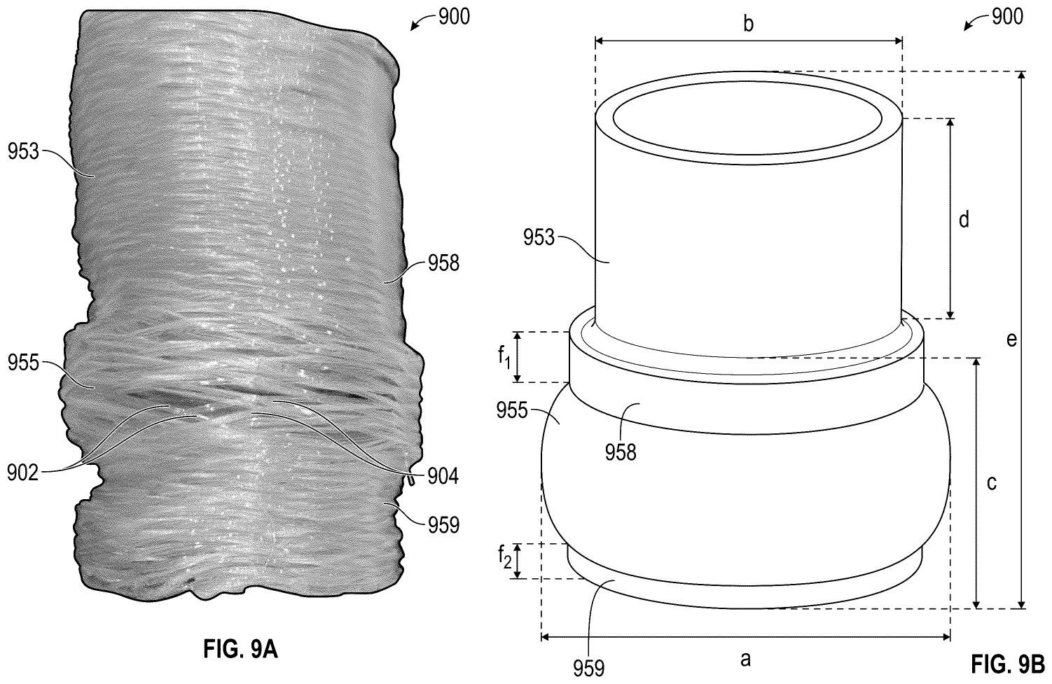

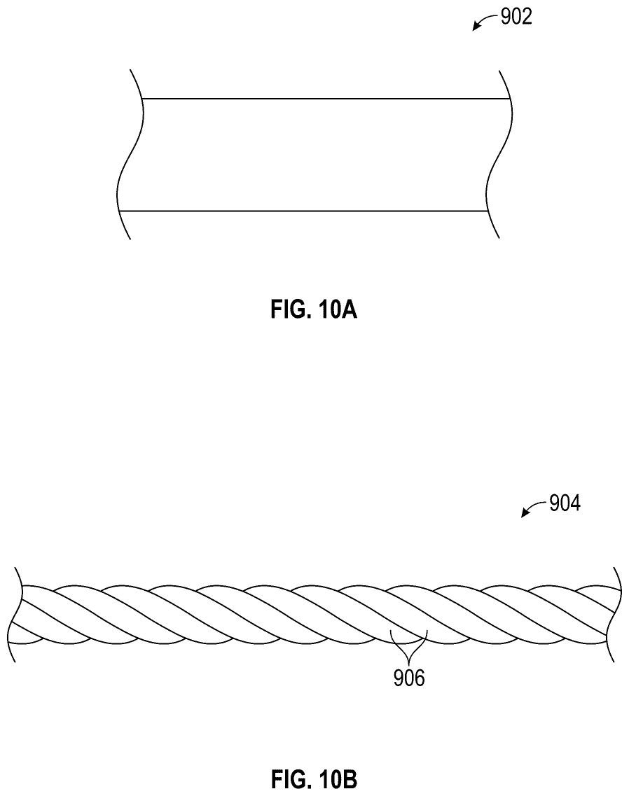

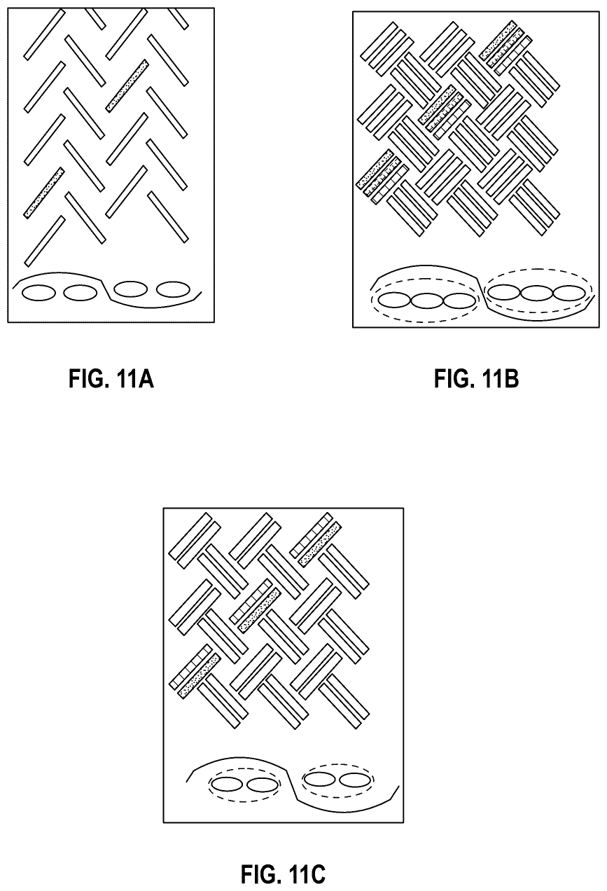

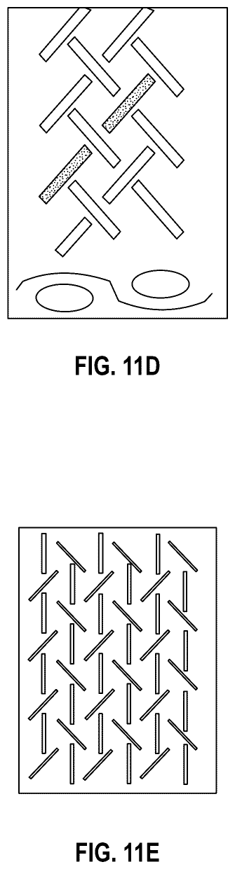

Turning to FIGS. 12-20B, exemplary methods and stages of production for generating a cylindrical braided material shown and described. FIG. 12 illustrates a high level method 1200 for manufacture or production of a multifilament and monofilament cylindrical braided material for a sealing member. As can be seen therein, a first phase 1202 includes generating the cylindrical braid (one exemplary method for the first phase 1202 is described in detail below with respect to FIG. 13), a second phase 1204 includes performing first and second heat setting (one exemplary method for the second phase 1204 is described in detail below with respect to FIG. 14), and a third phase 1206 includes performing post-processing to seal or fuse edges of the cylindrical braided structure (one exemplary method for the third phase 1206 is described in detail below with respect to FIG. 15).

Turning to FIG. 13, an exemplary method 1300 for generating a multifilament and monofilament cylindrical braided material is shown and described. At step 1302, multifilament and monofilament threads or strands are selected. As discussed above, in some examples, the monofilament thread can be PET. In some examples, the monofilament thread can be a different material, such as, for example, PEEK, TPU, polyesters (such as, PLA, P4HB, PGA, PCL, etc), polyamides, UHMWPE (Polyethylenes), or polypropylene). In some examples, the monofilament thread can have a selected weight, width, diameter, or gauge. In some examples, the multifilament thread can be PET. In some examples, the multifilament filament thread can be a different material, such as, High Tenacity PET, polyesters (such as, PLA, P4HB, PGA, etc), polyamides, UHMWPE (polyethylenes), polypropylene. In some examples, the multifilament filament thread can have a selected weight, width, diameter, or gauge. The multifilament thread can additionally have a selected number of individual filaments making up the multifilament thread and/or can have a selected weight, width, diameter, or gauge of the individual filaments.

Per step 1304, the monofilament and multifilament threads or yarns are loaded into a braider apparatus (for example, Steeger HS80/48). To manufacture or generate a cylindrical braided material comprising the monofilament PET strands and the multifilament PET strands, in some examples, each of the monofilament and multifilament threads can be loaded, spooled, or wound onto a separate bobbin. In some examples, the separate loading can enable the monofilament PET thread and the multifilament PET thread to be unwound from each bobbin contemporaneously and can result in monofilament PET strands and the multifilament PET strands moving more freely relative to each other within the resulting braided structure. Further, in some examples, the resulting cylindrical braided material can allow for greater expansion and/or preservation of bulk of the multifilament strands, and result in greater overall coverage and/or sealing of pores (spaces between the overlapping strands) within the cylindrical braided material.

In other examples, the monofilament and multifilament threads can be loaded, spooled, or wound onto a common bobbin (for example, the multifilament PET can be loosely wrapped around the monofilament PET and wound onto a common bobbin). In some examples, the common loading can enable the monofilament PET thread and the multifilament PET thread to be unwound simultaneously from the bobbin during braiding of the cylindrical material. In some examples, the common loading of the monofilament PET and multifilament PET threads can have operative advantages, such as creating more uniform tension on the wrapped strands, and can result in fewer breaks of the threads during the braiding process.

In examples including a triaxial braid pattern, an additional monofilament thread (PET or other material, such as, for example, PEEK, TPU, polyesters (such as, PLA, P4HB, PGA, PCL, etc), polyamides, UHM WPE (Polyethylenes), or polypropylene) can be loaded or spooled onto a separate bobbin.

End regions of the monofilament PET thread and the multifilament PET thread can be fed into the braider apparatus. Per step 1306, settings on the braider apparatus are input into the braider and can be customized or specific to the selected monofilament and multifilament threads and the desired braid configuration (such as those illustrated in FIGS. 11A-11E). In some examples, settings input or adjusted on the braider apparatus can include one or more of a selected braid pattern, tension on each bobbin, braiding speed, carrier spring diameter, braiding cone distance from braid, picks per inch (ppi) (defined as the number of times one strand crosses the braid shaft per each inch along the length of the final braid), a number of carriers or carrier count (defined as the number of carriers on the braiding table equipped to carry a yarn end around the mandrel to form the braid), a number of individual strand ends attached to each carrier, and/or other braider apparatus parameters. For example, one of the braid patterns illustrated in FIGS. 11A-11E or variations thereof or other braid patterns can be selected. In another example, a carrier count can be selected in a range of 8 to 192 carriers. In another example, a pick density can be set in a range of 15 ppi to 200 ppi.

The braiding parameters can be selected to result in a desired or targeted braid angle, for example, a braid angle in a range of 5 degrees to 85 degrees (as measured in an upwards direction from a central axis of one strand relative to a central axis of another strand in a plane of the braid, such as, for example, an angle between one of the multifilament strands and an intersecting one of the monofilament strands). In some examples, the braid angle cab be utilized to confirm proper replication on a different braiding machine or using a different braider set up.

It will be appreciated that the above parameters can be set or selected depending on an application of the braided cylindrical material and/or the sealing member. For example, coverage of a braided cylindrical material can be defined as the area covered by the multifilament and monofilament strands compared to the total surface area of the braid, measured as 100% minus the overall percentage of combined area of detectable pores out of the total surface area. In one specific example, percent braid coverage can be represented as:

$$Theoretical Percent Braid Coverage = ( 2 \cdot F - {F}^{2} ) \times 100$$

$$F = N \cdot P \cdot d / sin α$$

$$α = tan - 1 [ 2 \cdot π \cdot ( D + 2 \cdot d ) ( P / C ) ]$$

where a=braid angle, D=diameter under the braid, C=number of carriers, d=braid strand diameter, P=ppi, and N=Number of individual wires (ends) in each carrier. In another example, porosity of a braided cylindrical material (which can be a function of coverage) can be defined as the combined area of exposed pores compared to the total surface area of the braid, measured as the overall percentage of combined area of detectable pores out of the total surface area. In some examples, porosity of the exemplary sealing members disclosed herein can be in a range of 0% to 75%. In some examples, where a greater degree of sealing is desired, porosity of the sealing members can be in a range of 0% to 50% (such as, for the sealing members 356, 456).

In some examples, where an increased coverage and/or a decreased porosity of the sealing member are desired (for example, where increased cushioning qualities and/or fluid blocking qualities are desired for an implantable device), the aforementioned parameters can be selected to generate increased coverage and/or decreased porosity. In one specific example, a ppi of the braided cylindrical material can be increased to increase coverage and/or decrease porosity of a sealing member. In some examples, where a lower overall volume or mass of a sealing member are desired (for example, where a reduced overall crimp profile of an implantable device for transcatheter delivery is desired), the aforementioned parameters can be selected to generate decreased coverage and/or increased porosity. In one specific example, a ppi of the braided cylindrical material can be decreased to reduce an overall volume or mass of a sealing member.

Similarly, in some examples, carrier count can be either increased or decreased to respectively increase or decrease coverage. In some examples, the larger the diameter of a cylindrical braided material, a lager the carrier count may be implemented in order to generate the braided material.

Further, in some examples, braid pattern can be selected based on an application of the cylindrical braided material. For example, for increased cushioning and/or fluid blocking of a sealing member, a full diamond braid pattern can generate greater coverage than a half diamond. In another example, for improved crimping of a sealing member, a half diamond braid pattern can provide high stability and be resistant to strand or yarn pull-out given that it has the highest number of interlacement points and the shortest float length. In some examples, a targeted braid angle can be selected to result in a desired level of stiffness and/or radial expansion capabilities of sealing member. For example, a lower braid angle can result in a stiffer braid that is harder to deform. In another example, a higher braid angel can result in a more flexible braid that is more easily deformed.

Per step 1308, a first mandrel can be selected for braiding of the multifilament and monofilament strands thereon. In some examples, the first mandrel can be a generally straight or linear mandrel having a cylindrical shape. In some examples, the first mandrel can comprise steel. In some examples, the first mandrel can comprise a different material, such as, for example polytetrafluoroethylene (PTFE). In some examples, an OD of the first mandrel can be in a range of 1 mm to 32 mm. In some examples, at step 1310, a liner sheet (such as, a TPU sheet) can be disposed on a surface of the first mandrel so that the cylindrical material can be braided over the sheet, the liner sheet can form a liner of a sealing member. Per step 1312, the braid can then be generated onto or over the first mandrel (and over the liner material sheet, if included) via the braider apparatus.

In some examples, a TPU liner can be formed via one or more of: electrospinning, three-dimensional printing, spray coating (for example, ultrasonic spray coating), dip coating, lamination, and/or braiding. Materials that can be utilized for forming a TPU liner can include thermoplastic polyurethane (TPU), and/or TPU modified with siloxanes, fluorine, and/or other biocompatible additives. In some examples, polymers can be combined and/or blended. For example, polymers and various siloxanes can be combined or blended. For example, a copolymer of polycarbonate TPU with siloxane or a blend of silica particles with TPU can be utilized as materials to form a liner.

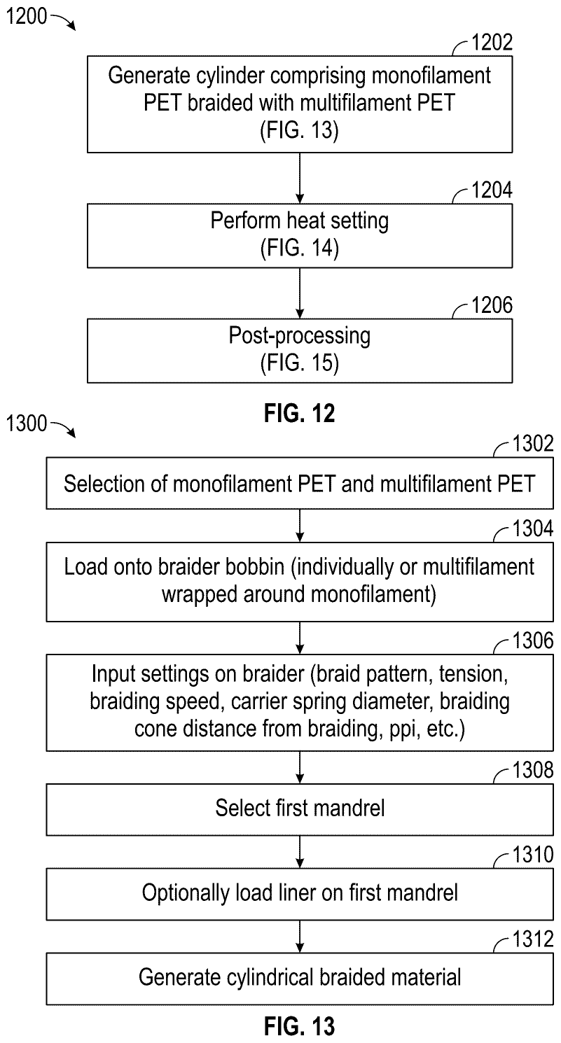

FIG. 16 shows an exemplary braided cylindrical material 1600 braided onto a first mandrel (not shown). As can be seen therein, the multifilament and monofilament strands are in a tightly wound or under-tension state after braiding onto the first mandrel.

A method 1400 for heat setting of the cylindrical braided material after its formation on the first mandrel is shown in FIG. 14. After formation of the braid on the first mandrel, at step 1402, the cylindrical material can be shape set in a first heat setting step on the first mandrel. In some examples, the first heat setting step is a soft heat setting step. In some examples, a soft heat set can include exposure of a polymer construct to a temperature at the lower end of its glass transition range. In this state, the polymer can become softer and more rubbery, allowing it to be manipulated enough to be transferred to and to better conform to a differently sized or shaped mandrel. For example, the first mandrel and the braided cylindrical material disposed thereon monofilament and multifilament PET strands can be incubated in an oven at a temperature in a range of 160° C. to 210° C. for a first period of time. In some examples, the first period of time is in a range of 3 min to 30 min. In one specific example, the first mandrel and the braided cylindrical material disposed thereon are incubated at 180° C. for ten minutes.

In some examples, the soft heat set can include a lower temperature relative to a subsequent (second) heat setting step (discussed below). In some examples, the soft heat setting step can be selected or designed to be at about a lower end of a glass transition range (Tg) of the multifilament strands (for example, slightly below, at, or slightly above a lower end of a glass transition range of the multifilament strands). In some examples, the soft heat setting step can be selected or designed to enable heat setting or relaxing of the multifilament strands.

As noted above with respect to FIG. 16, when braided onto the first mandrel the strands can be under tension (for example, under the selected tension of the braiding apparatus). In some examples, the braiding tension can result in a tight or axially elongated configuration for the overall structure of the cylindrical braided material (as illustrated in FIG. 16). In some examples, such tension can additionally or alternatively result in axial elongation of the multifilament strands and/or reduction space between the individual filaments of the multifilament strands. In some examples, the first heat step can function to loosen or relax the individual filaments within the multifilament strand so that they become more spaced apart relative to each other and/or return to an initial (pre-braiding) configuration of the multifilament strand. In some examples, the first heat step can also function to loosen or relax the monofilament strands and multifilament strands in the braid structure relative to each other.

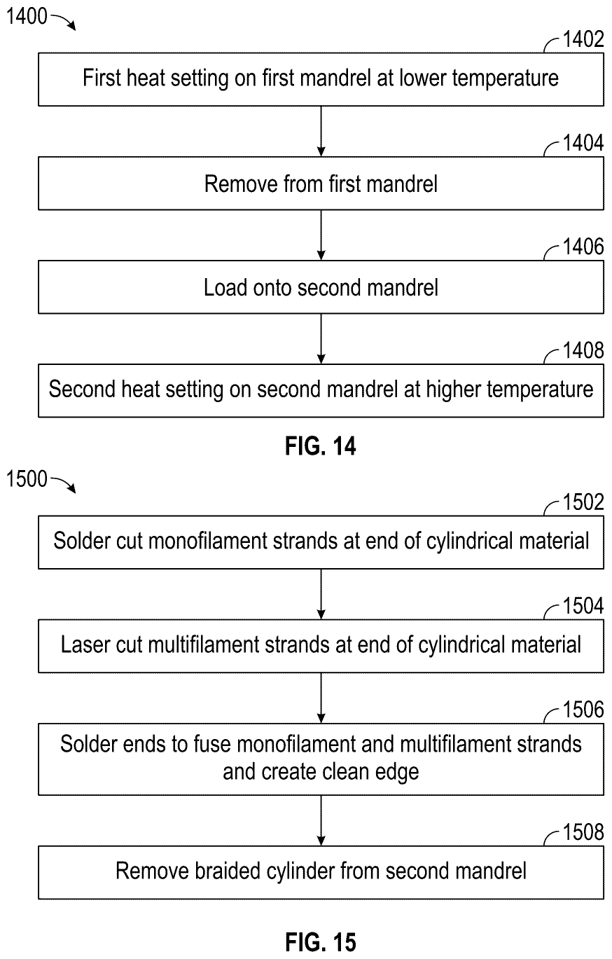

Per step 1404, after the first heat setting step, the cylindrical braided material can be removed from the first mandrel. FIG. 17 shows an exemplary braided cylindrical material 1700 removed from the first mandrel. As can be seen therein, the multifilament and monofilament strands are in a loosened or relaxed state relative to each other, and the cylindrical braided material has an overall linear cylindrical shape. Although not specifically illustrated, the individual filaments in the multifilament strands can also be in a loosened or relaxed state relative to each other.

After the first heat setting step and removal from the first mandrel, the cylindrical material can be transferred from the first mandrel to a second mandrel (step 1406). In some examples, a shape of the second mandrel can be selected or designed to correspond to a desired overall shape of a sealing member. In some examples, the second mandrel can have a non-linear cylindrical shape. For example, the second mandrel can include an extended or bulging region and a linear cylindrical region, which can correspond to, for example, the bulging portions 355, 455 of the sealing members 356, 456 shown in FIGS. 7 and 8. In another example, the second mandrel can include one or more tapered regions at opposing ends of a linear cylindrical region, which can correspond to, for example, the tapered proximal and distal ends 244p, 244d of the sealing member 244 shown in FIG. 6. In other examples the second mandrel have a different shape, for example, a linear cylindrical shape or have a shape including multiple bulging regions. In some examples, the second mandrel can comprise steel. In some examples, the second mandrel can comprise a different material, such as, for example polytetrafluoroethylene (PTFE). In some examples, an OD of the second mandrel can be in a range of 1 mm to 32 mm.

Per step 1408, after loading onto the second mandrel, the braided cylindrical material the second mandrel can be shape set in a second heat setting step on the second mandrel. In some examples, the second heat setting step is a hard heat setting step. A hard heat set can include exposure of a polymer to a temperature at the higher end of its glass transition range. Upon cooling from this temperature, the inner polymer structure becomes more crystalline, locking in the macro structure of the yarns and the braid itself to hold the shape the sample was set to while heated. For example, the second mandrel and a braided cylindrical material disposed thereon including monofilament and multifilament PET strands can be incubated in an oven at a temperature in a range of 160° C. to 210° C. for a second period of time. In some examples, the second period of time is in a range of 3 min to 30 min. In one specific example, the second mandrel and the braided cylindrical material disposed thereon are incubated at 210° C. for ten minutes.

In some examples, the hard heat setting can include a higher temperature relative to a first (soft) heat setting step (discussed above). In some examples, the hard heat setting step can be selected or designed to be at about a temperature at the higher end of its glass transition range of the monofilament strands (for example, slightly below, at, or slightly above a temperature at the higher end of its glass transition range of the monofilament strands). In some examples, the hard heat setting step can be selected or designed to enable shape setting of the monofilament strands and shape setting of the overall structure of the cylindrical braided material. In some examples, the second heat setting step creates a resilient form or shape for the cylindrical braided material, such that when the cylindrical braided material is axially elongated or compressed, it can return to and be biased toward the heat-set form or shape.

It will be appreciated that the dwell time or the duration of each of the first heat setting step and the second heat setting step can be selected based on the characteristics of the mandrel on which either the soft heat set or hard heat set is being performed. For example, solid mandrels or mandrels having a large wall thickness can require a longer dwell time to reach the glass transition temperature of the polymer which is braided over it, whereas thinner wall mandrels can require a shorter dwell time.

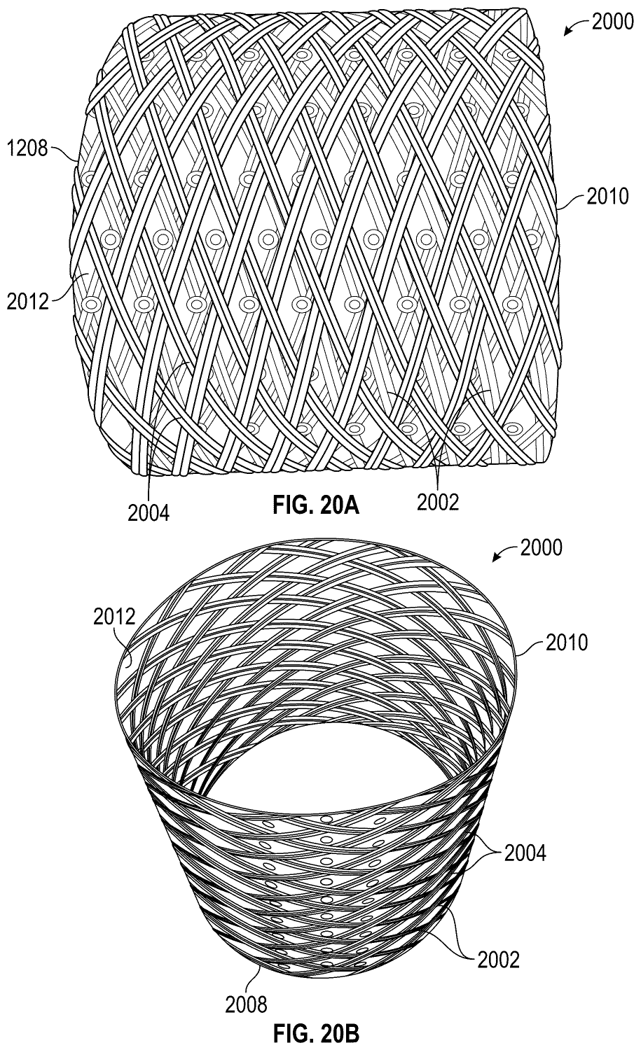

If a TPU liner is included, the cylindrical braided material can undergo a lamination step (discussed further below with respect to FIGS. 20A and 20B) before or after the hard heat setting step.

FIG. 15 shows an exemplary method 1500 for post-processing of the cylindrical braided material that can be carried out after the forgoing heat setting steps. At step 1502, the monofilament strands can be solder cut at each of the opposing ends of the cylindrical braided material. After the initial soldering step, the multifilament strands can be laser cut at each of the opposing ends of the cylindrical braided material (step 1506). In a second soldering step 1508, monofilament strands and the multifilament strands can be fused together along the circumferential edge of each the opposing ends of the cylindrical braided material. Fusion of the monofilament strands and the multifilament strands and the ends of the cylindrical braided material can prevent unraveling of the braid after being cut, and can enable the cylindrical braided material to be removed from the second mandrel (step 1508). In some examples, fusion of the monofilament strands and the multifilament strands can create a generally smooth edge at the ends of the cylindrical material. In some examples, the monofilament and multifilament strands can alternatively or additionally be cut with a blade apparatus. In some examples, the circumferential edge of each the opposing ends of the cylindrical braided material can be additionally or alternatively sealed or fused by exposure to a flat heating element, such as a hot plate, at a temperature in a range of 210° C. to 270° C.

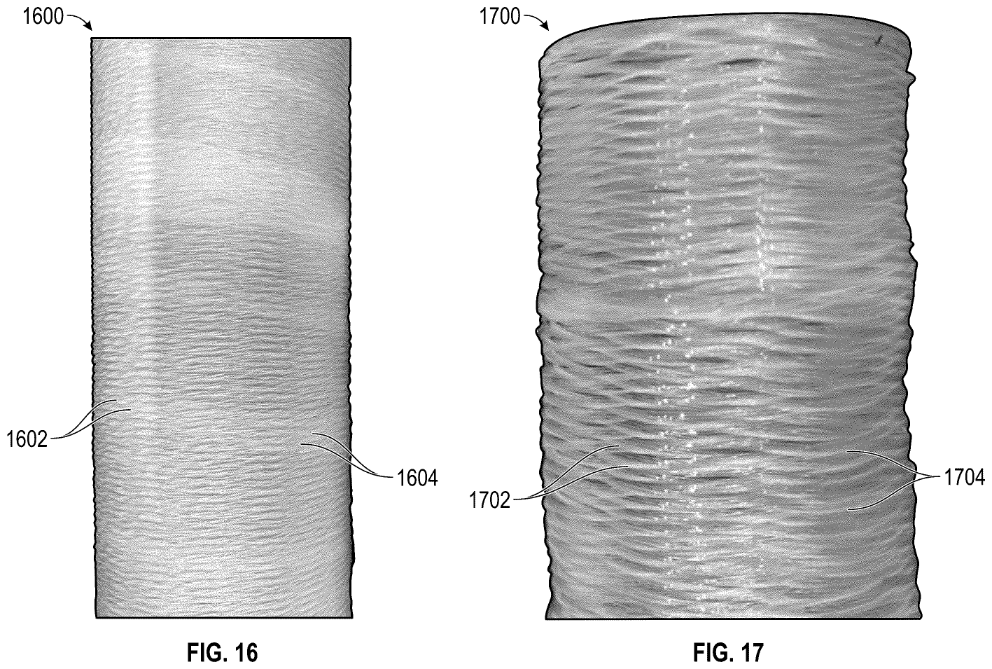

FIG. 18 shows an exemplary cylindrical braided material 1800 removed from the second mandrel. As can be seen therein, after removal from the second mandrel the cylindrical braided material 1800 is shape set to have a generally cylindrical shape including a non-bulging portion 1853 and a bulging region 1855 (similar to configuration of the cylindrical braided material 900 illustrated in FIGS. 9A and 9B, discussed above). The monofilament strands 1802 and the multifilament strands can be fused at first and second edges 1808, 1810 of the cylindrical braided material 1800. The cylindrical braided material 1800 can have a diameter a₁ at a widest section of the bulging portion 1855, and can have a diameter b₁ at the non-bulging portion 1853. Further, the cylindrical braided material 1800 can have an overall height e₁.

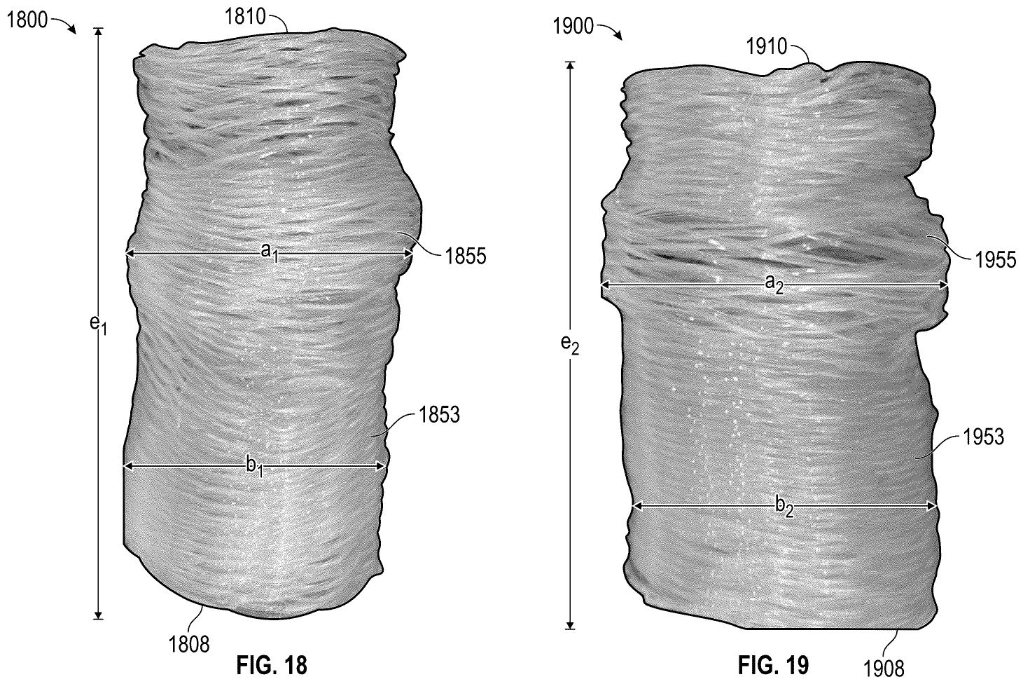

FIG. 19 shows an exemplary cylindrical braided material 1900 removed from the second mandrel. As can be seen therein, after removal from the second mandrel the cylindrical braided material 1900 is shape set to have a generally cylindrical shape including a non-bulging portion 1953 and a bulging region 1955 (similar to configuration of the cylindrical braided material 900 illustrated in FIGS. 9A and 9B, discussed above). The monofilament strands 1902 and the multifilament strands can be fused at first and second edges 1908, 1910 of the cylindrical braided material 1900. The cylindrical braided material 1900 can have a diameter a₂ at a widest section of the bulging portion 1955, and can have a diameter b₂ at the non-bulging portion 1953. Further, the cylindrical braided material 1900 can have an overall height e₂.

As can be seen in FIGS. 18 and 19, the cylindrical braided materials 1800, 1900 have different dimensions relative to each other, and can be, for example, generated to be fitted to differently sized or configured implantable devices. For example, the cylindrical braided material 1800 has a greater height than the cylindrical braided material 1900 (e₁>e₂), but is narrower relative to the cylindrical braided material 1900 (a₂>a₁ and b₂>b₁). Accordingly, in some examples, the cylindrical braided material 1800 can be configured to be a component (sealing member) of a relatively longer and narrower implantable device, while the cylindrical braided material 1900 can be configured to be a component (sealing member) of a relatively shorter and wider implantable device. In other examples, the cylindrical braided materials 1800, 1900 can be configured to be a component (sealing member) of similarly sized implantable devices, but which may be targeted for different applications, such as being configured for different locations of implantation (for example, one implantable device configured for implantation in an aortic heart valve and another implantable device configured for implantation in a mitral heart valve).

It will be appreciated that the dimensions, shape, braid pattern, materials and diameters of the multifilament and monofilament strands, as well as braider apparatus parameters (such as tension on each bobbin, braiding speed, carrier spring diameter, braiding cone distance from braid, ppi, carrier count, strand ends attached to each carrier, braid angle, and/or other braider apparatus parameters) can be selected to generate a cylindrical braided material configured for a specific or selected application or to be a component (sealing member) of a specified or selected implantable device.

Further, as discussed above, in some examples, a cylindrical braided material can be formed over a liner material sheet disposed on the first mandrel, which can comprise TPU, FEP, siloxanes, and/or acrylic and can be used to form a liner of the cylindrical braided material. A liner may be utilized for applications or implantable devices where additional structural stability of the sealing member is desired.

FIGS. 20A and 20B show an exemplary cylindrical braided material 2000 including a TPU liner 2012 removed from the mandrel. As can be seen therein, the cylindrical braided material 2000 has a shape that lacks any bulging regions. The TPU liner 2012 can provide an underlying support structure for the braided monofilament and multifilament strands 2002, 2004. Accordingly, in some examples, the opposing edges 2008, 2010 of the cylindrical braided material 2000 are unfused and the shape and braiding of the multifilament strands 2002, 2004 is retained by fusion or attachment to the TPU liner 2012. In other examples, the edges of the cylindrical braided material 2000 can additionally be fused.

In some examples, the method of generating the cylindrical braided material including the TPU liner can additionally include a lamination step when disposed on the first mandrel or the second mandrel. The lamination step can comprise incubating the cylindrical braided material and the mandrel in an oven at a temperature in a range of 25° C. to 230° C., at a pressure in a range of 0.5 M Pa to 15 M Pa for a period of time within a range of 20 sec to 600 sec. In some examples, after the lamination step, the cylindrical braided material can go through post-processing steps without a second heat setting step. In other examples, the second heat setting step on a second mandrel can be performed.

After generation and post-processing of a cylindrical braided material, it can be attached to an implantable device and can function as, for example, a sealing member thereof. In some examples, the cylindrical braided material can have a sufficient suture retention strength to limit or prevent any strands from breaking, coming loose, and/or separating from the implantable device. For example, the cylindrical braided material can have a suture retention strength that is in a range of 5 N to 25 N, such as, for example, in a range of 10 N to 11 N.

The cylindrical braided material including the monofilament strands braided with the multifilament strands can be a single layer that provides both structural integrity and/or resiliency, as well as bulk, coverage, and/or softness of the sealing member. Thus, the cylindrical braided material can eliminate the need for alignment and attachment (suturing) of a first layer configured to provide structural integrity/resiliency and a second layer configured to provide softness/bulk, as in conventional sealing members. Thus, in some examples, the cylindrical braided materials and sealing members disclosed herein can result in reduced failure and reduced production time and costs for an implantable device including the sealing members relative to conventional sealing members.

### Sterilization

It will be appreciated that any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples heat/thermal sterilization include steam sterilization and autoclaving. Examples of radiation for use in sterilization include gamma radiation, ultra-violet radiation, and/or electron beam. Examples of chemicals for use in sterilization include ethylene oxide, hydrogen peroxide, peracetic acid, formaldehyde, and/or glutaraldehyde. Sterilization with hydrogen peroxide may be accomplished using hydrogen peroxide plasma, for example.

### Treatment Techniques and Methods

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above described examples of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An implantable device comprising: an annular frame comprising a plurality of interconnected struts; and a sealing member disposed on an exterior surface of the annular frame, the sealing member comprising a plurality of monofilament strands braided with a plurality of multifilament strands to form a cylindrical braided material.

Example 2. The implantable device of any example disclosed herein, particularly example 1, wherein the implantable device is a prosthetic heart valve and further comprises a valvular structure disposed in an interior of the annular frame.

Example 3. The implantable device of any example disclosed herein, particularly example 1, wherein the implantable device is a stent.

Example 4. The implantable device of any example disclosed herein, particularly examples 1-3, wherein the monofilament strands comprise monofilament PET strands.

Example 5. The implantable device of any example disclosed herein, particularly examples 1-4, wherein the multifilament strands comprise multifilament PET strands.

Example 6. The implantable device of any example disclosed herein, particularly examples 1-5, wherein the sealing member is a single layer comprising the cylindrical braided material.

Example 7. The implantable device of any example disclosed herein, particularly examples 1-5, wherein the sealing member comprises a first layer comprising the cylindrical braided material and a second layer comprising an interior liner.

Example 8. The implantable device of any example disclosed herein, particularly example 7, wherein the interior liner is a TPU liner.

Example 9. The implantable device of any example disclosed herein, particularly examples 1-8, wherein the cylindrical braided material comprises a braid pattern comprising one of a regular, one up-two down braid pattern; a Hercules, three up-three down braid pattern; a diamond or full load, two up-two down braid pattern; or a half diamond, one up-one down braid pattern.

Example 10. The implantable device of any example disclosed herein, particularly examples 1-8, wherein the cylindrical braided material comprises a regular braid pattern including a triaxial thread, and wherein the triaxial thread is a monofilament thread.

Example 11. The implantable device of any example disclosed herein, particularly examples 1-10, wherein the sealing member comprises a relaxed configuration including a bulging portion and a linear portion.

Example 12. The implantable device of any example disclosed herein, particularly example 11, wherein the monofilament strands of the cylindrical braided material are shape-set to form the bulging portion and the linear portion of the sealing member in the relaxed configuration.

Example 13. The implantable device of any example disclosed herein, particularly example 12, wherein the sealing member is moveable between an axially elongated configuration and the relaxed configuration.

Example 14. The implantable device of any example disclosed herein, particularly example 13, wherein the shape-set monofilament strands bias the sealing member toward to the relaxed configuration.

Example 15. The implantable device of any example disclosed herein, particularly examples 1-14, wherein the multifilament strands are configured to provide one or more of bulk or coverage for the sealing member.

Example 16. The implantable device of any example disclosed herein, particularly examples 1-15, wherein the sealing member is configured to, when the implantable device is implanted within a native vein, artery, or heart valve, contour to anatomy disposed adjacent to the implantable device and resist or limit leakage of blood between the anatomy and the implantable device.

Example 17. The implantable device of any example disclosed herein, particularly examples 1-16, wherein the sealing member is configured to, when the implantable device is implanted within a native vein, a native artery, or a native heart valve, contour to anatomy disposed adjacent to the implantable device and anchor the implantable device to the anatomy.

Example 18. The implantable device of any example disclosed herein, particularly examples 1-17, wherein the cylindrical braided material has a pick density in a range of 15 ppi to 200 ppi.

Example 19. The implantable device of any example disclosed herein, particularly examples 1-18, wherein the cylindrical braided material has a braid angle of the multifilament strands relative to intersecting ones of the monofilament strands in a range of 15 ppi to 200 ppi.

Example 20. The implantable device of any example disclosed herein, particularly examples 1-19, wherein the cylindrical braided material has a percent braid coverage in a range of 25% to 100%.

Example 21. The implantable device of any example disclosed herein, particularly examples 1-20, wherein the cylindrical braided material has a percent porosity in a range of 0% to 75%.

Example 22. The implantable device of any example disclosed herein, particularly examples 1-21, wherein the multifilament strands are texturized.

Example 23. The implantable device of any example disclosed herein, particularly examples 1-22, wherein a diameter of each of the monofilament strands in a range of 0.002 in to 0.015 in.

Example 24. The implantable device of any example disclosed herein, particularly examples 1-23, wherein a diameter of each of the multifilament strands in a range of 0.0015 in to 0.004 in.

Example 25. The implantable device of any example disclosed herein, particularly examples 1-24, wherein a diameter of each filament of the multifilament strands in a range of 0.002 mm to 0.005 mm.

Example 26. An implantable docking device configured for securing a prosthetic valve, the implantable docking device comprising: a coil member configured to be transitioned from a delivery configuration to a coiled configuration, wherein the coil member, when in the coiled configuration, comprises a plurality of helical turns; and a sealing member extending, relative to a central longitudinal axis of the coil member in the coiled configuration, circumferentially over at least a portion of each of one or more of the helical turns; wherein the sealing member comprises a plurality of monofilament strands braided with multifilament strands to form a cylindrical braided material.

Example 27. The implantable docking device of any example disclosed herein, particularly example 26, wherein the monofilament strands comprise monofilament PET strands.

Example 28. The implantable docking device of any example disclosed herein, particularly examples 26 or 27, wherein the multifilament strands comprise multifilament PET strands.

Example 29. The implantable docking device of any example disclosed herein, particularly examples 26-28, wherein the sealing member is a single layer comprising the cylindrical braided material.

Example 30. The implantable docking device of any example disclosed herein, particularly examples 26-28, wherein the sealing member comprises a first layer comprising the cylindrical braided material and a second layer comprising an interior liner.

Example 31. The implantable docking device of any example disclosed herein, particularly example 30, wherein the interior liner is a TPU liner.

Example 32. The implantable docking device of any example disclosed herein, particularly examples 26-31, wherein the cylindrical braided material comprises a braid pattern comprising one of a regular, one up—two down braid pattern; a Hercules, three up—three down braid pattern; a diamond or full load, two up—two down braid pattern; or a half diamond, one up—one down braid pattern.

Example 33. The implantable docking device of any example disclosed herein, particularly examples 26-31, wherein the cylindrical braided material comprises a regular braid pattern including a triaxial thread, and wherein the triaxial thread is a monofilament thread.

Example 34. The implantable docking device of any example disclosed herein, particularly examples 26-33, wherein the sealing member comprises a relaxed configuration including a central portion, a tapered proximal end region, and a tapered distal end region.

Example 35. The implantable docking device of any example disclosed herein, particularly example 34, wherein the monofilament strands of the cylindrical braided material are shape-set to form the central portion, the tapered proximal end region, and the tapered distal end region of the sealing member in the relaxed configuration.

Example 36. The implantable docking device of any example disclosed herein, particularly example 35, wherein the sealing member is moveable between an axially elongated configuration and the relaxed configuration.

Example 37. The implantable docking device of any example disclosed herein, particularly example 36, wherein the shape-set monofilament strands bias the sealing member toward to the relaxed configuration.

Example 38. The implantable docking device of any example disclosed herein, particularly examples 26-37, wherein the multifilament strands are configured to provide one or more of bulk or coverage for the sealing member.

Example 39. The implantable docking device of any example disclosed herein, particularly examples 26-38, wherein the sealing member is configured to, when the implantable docking device is implanted within a native heart valve, contour to anatomy disposed adjacent to the implantable docking device and resist or limit leakage of blood between the anatomy and the implantable docking device.

Example 40. The implantable docking device of any example disclosed herein, particularly examples 26-39, wherein the sealing member is configured to, when the implantable docking device is implanted within a heart valve, contour to anatomy disposed adjacent to the implantable docking device and anchor the implantable docking device to the anatomy.

Example 41. The implantable docking device of any example disclosed herein, particularly examples 26-40, wherein the cylindrical braided material has a pick density in a range of 15 ppi to 200 ppi.

Example 42. The implantable docking device of any example disclosed herein, particularly examples 26-41, wherein the cylindrical braided material has a braid angle of the multifilament strands relative to intersecting ones of the monofilament strands in a range of 15 ppi to 200 ppi.

Example 43. The implantable docking device of any example disclosed herein, particularly examples 26-42, wherein the cylindrical braided material has a percent braid coverage in a range of 25% to 100%.

Example 44. The implantable docking device of any example disclosed herein, particularly examples 26-43, wherein the cylindrical braided material has a percent porosity in a range of 0% to 75%.

Example 45. The implantable docking device of any example disclosed herein, particularly examples 26-44, wherein the multifilament strands are texturized.

Example 46. The implantable docking device of any example disclosed herein, particularly examples 26-45, wherein a diameter of each of the monofilament strands in a range of 0.002 in to 0.015 in.

Example 47. The implantable docking device of any example disclosed herein, particularly examples 26-46, wherein a diameter of each of the multifilament strands in a range of 0.0015 to 0.004 in.

Example 48. The implantable docking device of any example disclosed herein, particularly examples 26-47, wherein a diameter of each filament of the multifilament strands in a range of 0.002 mm to 0.005 mm.

Example 49. A sealing member for use with an implantable device, the sealing member comprising: a cylindrical braided material comprising a plurality of monofilament PET strands braided with multifilament PET strands.

Example 50. The sealing member of any example disclosed herein, particularly example 49, wherein the implantable device is a prosthetic heart valve and the sealing member is an outer skirt for the prosthetic heart valve.

Example 51. The sealing member of any example disclosed herein, particularly example 49, wherein the implantable device is a stent and the sealing member is an outer skirt for the stent.

Example 52. The sealing member of any example disclosed herein, particularly example 49, wherein the implantable device is a docking device configured secure a prosthetic heart valve and the sealing member is a paravalvular leakage guard for the docking device.

Example 53. The sealing member of any example disclosed herein, particularly examples 49-52, wherein the sealing member is a single layer structure.

Example 54. The sealing member of any example disclosed herein, particularly examples 49-52, wherein the cylindrical braided material is a first layer and the sealing member further comprises a second layer that is an interior liner of the cylindrical braided material.

Example 55. The sealing member of any example disclosed herein, particularly example 54, wherein the interior liner is a TPU liner.

Example 56. The sealing member of any example disclosed herein, particularly examples 49-55, wherein the cylindrical braided material comprises a braid pattern comprising one of a regular, one up-two down braid pattern; a Hercules, three up-three down braid pattern; a diamond or full load, two up-two down braid pattern; or a half diamond, one up-one down braid pattern.

Example 57. The sealing member of any example disclosed herein, particularly examples 49-55, wherein the cylindrical braided material comprises a regular braid pattern including a triaxial thread, and wherein the triaxial thread is a monofilament thread.

Example 58. The sealing member of any example disclosed herein, particularly examples 49-57, wherein the sealing member comprises a relaxed, radially expanded configuration, and the monofilament PET strands of the cylindrical braided material are shape-set to bias the sealing member towards the relaxed, radially expanded configuration.

Example 59. The sealing member of any example disclosed herein, particularly examples 49-58, wherein the multifilament strands are configured to provide one or more of bulk or coverage for the sealing member.

Example 60. The sealing member of any example disclosed herein, particularly examples 49-59, wherein the sealing member is configured to, when the implantable device is implanted within a native vein, artery, or heart valve, contour to anatomy disposed adjacent to the implantable device and resist or limit leakage of blood between the anatomy and the implantable device.

Example 61. The sealing member of any example disclosed herein, particularly examples 49-60, wherein the sealing member is configured to, when the implantable device is implanted within a native vein, a native artery, or a native heart valve, contour to anatomy disposed adjacent to the implantable device and anchor the implantable device to the anatomy.

Example 62. The sealing member of any example disclosed herein, particularly examples 49-61, wherein the cylindrical braided material has a pick density in a range of 15 ppi to 200 ppi.

Example 63. The sealing member of any example disclosed herein, particularly examples 49-62, wherein the cylindrical braided material has a braid angle of the multifilament strands relative to intersecting ones of the monofilament strands in a range of 15 ppi to 200 ppi.

Example 64. The sealing member of any example disclosed herein, particularly examples 49-63, wherein the cylindrical braided material has a percent braid coverage in a range of 50% to 100%.

Example 65. The sealing member of any example disclosed herein, particularly examples 49-64, wherein the cylindrical braided material has a percent porosity in a range of 0% to 50%.

Example 66. The sealing member of any example disclosed herein, particularly examples 49-65, wherein the multifilament strands are texturized.

Example 67. The implantable device of any example disclosed herein, particularly examples 49-66, wherein a diameter of each of the monofilament strands in a range of 0.002 in to 0.015 in.

Example 68. The implantable device of any example disclosed herein, particularly examples 49-67, wherein a diameter of each of the multifilament strands in a range of 0.0015 in to 0.004 in.

Example 69. The implantable device of any example disclosed herein, particularly examples 49-68, wherein a diameter of each filament of the multifilament strands in a range of 0.002 mm to 0.005 mm.

Example 70. A method of generating a sealing member, the method comprising: loading a multifilament PET thread and a monofilament PET thread into a braider apparatus; setting one or more braiding parameters of the braider apparatus; generating a cylindrical braided material on a first mandrel comprising the multifilament PET thread braided with the monofilament PET thread; incubating the cylindrical braided material on the first mandrel at a first temperature for a first period of time; transferring the cylindrical braided material to a second mandrel; incubating the cylindrical braided material at a second temperature for a second period of time; and removing the cylindrical braided material from the second mandrel.

Example 71. The method of any example disclosed herein, particularly example 70, wherein the first temperature is a lower temperature relative to the second temperature.

Example 72. The method of any example disclosed herein, particularly examples 70 or 71, further comprising soldering the monofilament PET thread at opposing ends of the cylindrical braided material to create a plurality of free ends of the monofilament PET thread.

Example 73. The method of any example disclosed herein, particularly example 72, further comprising laser cutting the multifilament PET thread at the opposing ends of the cylindrical braided material to create a plurality of free ends of the multifilament PET thread.

Example 74. The method of any example disclosed herein, particularly example 73, further comprising soldering the free ends of the monofilament PET thread and the free ends of the multifilament PET thread to create a fused edge at each of the opposing ends of the cylindrical braided material.

Example 75. The method of any example disclosed herein, particularly examples 70-74, further comprising loading TPU sheet on the first mandrel and laminating the cylindrical braided material with the TPU sheet to form a liner.

Example 76. The method of any example disclosed herein, particularly examples 70-75, wherein the setting one or more braiding parameters comprises selecting one or more of a selected braid pattern, a tension on each bobbin, a braiding speed, a carrier spring diameter, a braiding cone distance from braid, a picks per inch (ppi), a carrier count, a number of individual strand ends attached to each carrier, or a braid angle.

Example 77. The method of any example disclosed herein, particularly examples 70-76, wherein multifilament PET thread and a monofilament PET thread a loaded onto a common bobbin.

Example 78. The method of any example disclosed herein, particularly examples 70-76, wherein multifilament PET thread is loaded onto a first bobbin and a monofilament PET thread a loaded onto a second bobbin.

Example 79. The method of any example disclosed herein, particularly examples 70-78, wherein the first mandrel is a straight mandrel.

Example 80. The method of any example disclosed herein, particularly examples 70-79, wherein the second mandrel is a shaped mandrel.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An implantable device comprising:
an annular frame comprising a plurality of interconnected struts; and
a sealing member disposed on an exterior surface of the annular frame, the sealing member comprising a plurality of monofilament strands braided with a plurality of multifilament strands to form a cylindrical braided material.

2. The implantable device of claim 1, wherein the implantable device is a prosthetic heart valve and further comprises a valvular structure disposed in an interior of the annular frame.

3. The implantable device of claim 1, wherein the implantable device is a stent.

4. The implantable device of claim 1, wherein the monofilament strands comprise monofilament PET strands.

5. The implantable device of claim 1, wherein the multifilament strands comprise multifilament PET strands.

6. The implantable device of claim 1, wherein the sealing member comprises a first layer comprising the cylindrical braided material and a second layer comprising an interior liner.

7. The implantable device of claim 6, wherein the interior liner is a TPU liner.

8. The implantable device of claim 1, wherein the cylindrical braided material comprises a braid pattern comprising one of a regular, one up-two down braid pattern; a Hercules, three up-three down braid pattern; a diamond or full load, two up-two down braid pattern; or a half diamond, one up-one down braid pattern.

9. The implantable device of claim 1, wherein the cylindrical braided material comprises a regular braid pattern including a triaxial thread, and wherein the triaxial thread is a monofilament thread.

10. The implantable device of claim 1, wherein the sealing member comprises a relaxed configuration including a bulging portion and a linear portion.

11. The implantable device of claim 10, wherein the monofilament strands of the cylindrical braided material are shape-set to form the bulging portion and the linear portion of the sealing member in the relaxed configuration.

12. The implantable device of claim 1, wherein the sealing member is configured to, when the implantable device is implanted within a native vein, a native artery, or a native heart valve, contour to anatomy disposed adjacent to the implantable device and anchor the implantable device to the anatomy.

13. An implantable docking device configured for securing a prosthetic valve, the implantable docking device comprising:
a coil member configured to be transitioned from a delivery configuration to a coiled configuration, wherein the coil member, when in the coiled configuration, comprises a plurality of helical turns; and
a sealing member extending, relative to a central longitudinal axis of the coil member in the coiled configuration, circumferentially over at least a portion of each of one or more of the helical turns;
wherein the sealing member comprises a plurality of monofilament strands braided with multifilament strands to form a cylindrical braided material.

14. The implantable docking device of claim 13, wherein the monofilament strands comprise monofilament PET strands.

15. The implantable docking device of claim 13, wherein the multifilament strands comprise multifilament PET strands.

16. The implantable docking device of claim 13, wherein the sealing member comprises a relaxed configuration including a central portion, a tapered proximal end region, and a tapered distal end region.

17. The implantable docking device of claim 16, wherein the monofilament strands of the cylindrical braided material are shape-set to form the central portion, the tapered proximal end region, and the tapered distal end region of the sealing member in the relaxed configuration.

18. A method of generating a sealing member, the method comprising:
loading a multifilament PET thread and a monofilament PET thread into a braider apparatus;
setting one or more braiding parameters of the braider apparatus;
generating a cylindrical braided material on a first mandrel comprising the multifilament PET thread braided with the monofilament PET thread;
incubating the cylindrical braided material on the first mandrel at a first temperature for a first period of time;
transferring the cylindrical braided material to a second mandrel;
incubating the cylindrical braided material at a second temperature for a second period of time; and
removing the cylindrical braided material from the second mandrel.

19. The method of claim 18, wherein the first temperature is a lower temperature relative to the second temperature.

20. The method of claim 18, further comprising soldering the monofilament PET thread at opposing ends of the cylindrical braided material to create a plurality of free ends of the monofilament PET thread.
